(19) United States
(12) Patent Application Publication
(10) Pub. No.: US 2025/0275986 A1
(43) Pub. Date: Sep. 4, 2025

(54) COMBINATION OF A KETONE BODY OR KETOGENIC COMPOUND WITH AN ANALGESIC OR ANTIOXIDANT

(71) Applicant: KETOSWISS AG, Basel (CH)

(72) Inventor: Elena GROSS, Basel (CH)

(73) Assignee: KETOSWISS AG, Basel (CH)

(21) Appl. No.: 18/579,573

(22) PCT Filed: Jul. 17, 2022
(86) PCT No.: PCT/EP2022/069972

§ 371 Date: Jan. 16, 2024

(30) Foreign Application Priority Data
Jul. 17, 2021 (EP) 21186274.3

(51) Int. Cl. A61K 31/603; A61K 9/00; A61K 31/121; A61K 31/192; A61K 31/4045; A61K 45/06; A61P 25/06

(52) U.S. Cl. CPC A61K 31/603; A61K 9/0056; A61K 31/121; A61K 31/192; A61K 31/4045; A61K 45/06; A61P 25/06

(57) ABSTRACT

The present invention is in the field of pharmaceuticals, functional food products or supplements, and medical foods or food for special medical purposes (FSMP). In particular, the present invention relates to a combination of (i) a ketone body and/or a ketogenic compound, and (ii) an analgesic and/or an antioxidant, for use in treating and/or preventing pain such as headache, migraine and/or symptoms of migraine in a subject. Furthermore, the invention relates to a composition comprising (i) a ketone body and/or a ketogenic compound, and (ii) an analgesic and/or an antioxidant, such as a pharmaceutical composition, a food product, a food supplement, a nutritional aid, a medical food or food for special medical purposes (FSMP), and uses thereof.

The present invention is in the field of pharmaceuticals, functional food products or supplements, and medical foods or food for special medical purposes (FSMP). In particular, the present invention relates to a combination of (i) a ketone body and/or a ketogenic compound, and (ii) an analgesic and/or an antioxidant, for use in treating and/or preventing pain such as headache, migraine and/or symptoms of migraine in a subject. Furthermore, the invention relates to a composition comprising (i) a ketone body and/or a ketogenic compound, and (ii) an analgesic and/or an antioxidant, such as a pharmaceutical composition, a food product, a food supplement, a nutritional aid, a medical food or food for special medical purposes (FSMP), and uses thereof.

Migraine is a complex, genetically heterogeneous, common and debilitating neurological disorder that affects approximately 15% of the world population. With a peak incidence during the most productive years of life, migraine not only causes much suffering, but also inflicts substantial costs on society: approximately € 18.5 billion per year in Europe alone. It is characterized by recurrent moderate to severe, typically throbbing and unilateral, headache attacks that typically last 4 to 72 h. Often, the headaches are aggravated by any kind of physical activity, and accompanied by photo, phono-, and/or osmophobia, and/or nausea. Migraine can be divided into two major subgroups, based on the presence or absence of an aura, a phase of transient and reversible visual, sensory or motor disturbances that typically occurs up to one hour before the headache phase in one third of migraineurs. The headache (ictal) phase of a migraine attack is typically accompanied by neurological symptoms during a premonitory phase preceding the headache by up to 12 hours and a postdromal phase, which follows the migraine and can last hours or days.

Current migraine treatment options are limited and their mechanisms of action are also not completely understood. While the primary goals of preventative migraine treatment include reducing headache frequency and restoring function, an additional goal is the prevention of progression to chronic migraine. None of the prophylactic agents available to date (such as beta-blockers, anticonvulsants or antidepressants) are migraine-specific and most are associated with significant, often intolerable, side-effects. Furthermore, their migraine-preventive properties are moderate at most (<25% average reduction in migraine frequency).

A ketogenic diet or regular administration of exogeneous ketone bodies such as 3-hydroxybutyrate or precursors thereof (ketogenic compounds) have been shown to effectively prevent migraine attacks while being well-tolerable (WO 2018/115158).

The administration of a ketone body, i.e. β-hydroxybutyrate (βHB) and/or acetoacetate (AcAc), or a ketogenic compound can induce a state of ketosis which is a metabolic state in which some of the body's energy supply comes from ketone bodies in the blood, in contrast to a state of glycolysis, in which predominantly blood glucose provides energy. Ketosis commonly occurs during starvation or fasting. Ketosis is typically characterized by serum concentrations of ketone bodies, i.e. βHB and AcAc, over 0.5 mM. Induction of ketosis by administration of exogeneous ketone bodies or ketogenic compounds is advantageous over fasting and/or a ketogenic diet with a high fat, low carbohydrate and moderate protein content, because the patient adherence is much higher (see, e.g., WO2018/115158). Ketogenic compounds include, e.g., medium chain triglycerides (MCTs), 1,3-butanediol, triacetin, ketogenic amino acids, and ketone esters, for example, esters of βHB or AcAc with βHB, AcAc, alcohols such as 1,3-butanediol or glycerol or fatty acids (e.g. medium chain fatty acids), or esters of fatty acids (e.g. medium chain fatty acids) with alcohols such as 1,3-butanediol or glycerol, or combinations thereof such as esters of βHB and fatty acids with an alcohol such as 1,3-butanediol or glycerol (see, e.g., WO2010021766; US 2001/0014696; US 2015/0164855; U.S. Pat. No. 7,351,736; US 2019/0209491; Clarke (2012), Regul Toxicol Pharmacol. 63(3); Desrochers (1995), Am J Physiol, 268(4 Pt 1):E660-7; and Veech (2014), Journal of Lipid Research Vol. 55).

Thus, it is known in the art that ketone bodies and ketogenic compounds such as ketone esters are preventive drugs that are well suitable for the treatment of migraine, i.e., by preventing the occurrence or reducing the frequency of acute migraine attacks. However, it is entirely unclear, whether or how ketone bodies or ketogenic compounds interact with other drugs, and whether certain combinations thereof have any additional benefits for treating or preventing migraine or other headaches. It also remains unclear whether the administration of exogeneous ketone bodies or ketogenic compounds is useful for the instant treatment of an acute migraine attack or other acute headaches.

In particular, it is not known whether the uptake of ketone bodies or a ketogenic compound provides rapid effects in order to ameliorate, eliminate, or prevent the aggravation of acute headache or an acute migraine attack.

The treatment of acute migraine attacks or other acute headaches with analgesics is still unsatisfactory. Commonly used for this purpose are, for example, non-steroidal anti-inflammatory drugs (NSAIDs) such as ibuprofen, ketoprofen, acetylsalicylic acid, diclofenac, ketorolac, or the combination of acetaminophen (paracetamol), acetylsalicylic acid, and caffeine (see, e.g., Gilmore (2011), American Family Physician. 83 (3); Rabbie (2013), Cochrane Database Syst Rev (4); Derry (2013), Cochrane Database Syst Rev (4)); and Kirti (2013), Cochrane Database Syst Rev (4). However, ibuprofen, for example, has been found to provide effective pain relief in only about 50% of migraine attacks. The insufficient efficacy of these NSAIDs may require high doses thereof and/or treatment with stronger drugs such as triptans and ergotamines, which may lead to the development of medication overuse headache, in which the headaches become more severe and more frequent, and other undesired side-effects such as gastrointestinal, renal and/or liver damage or distress, nausea and/or tiredness. In many cases, an acute migraine attack or other acute headaches can currently not be treated at all with an acceptable side-effect profile, as a large proportion of patients does not respond to simple analgesics and/or triptans.

In addition, acute migraine management with the use of triptans or simple analgesics comes with the problem of rebound headaches, which is the reoccurrence of the migraine pain after the substance stops working, e.g. when it is metabolized or diluted out. As the migraine attack itself is still ongoing, further rescue medication intake is required, which further exacerbates the problem of medication overuse as well as medication overuse headache. Hence, current rescue migraine medication use is permitted on maximum of 10 days per months. For many higher-frequency migraine patients this means that they are without remedy on several days per months.

Thus, there is a still a need for improved means and methods for treating and/or preventing pain such as headache, migraine and/or symptoms of migraine.

The above technical problem is solved by the invention characterized in the claims and as described in the following.

Accordingly, the invention relates to a combination of (i) a ketone body and/or a ketogenic compound, and (ii) an analgesic and/or an antioxidant, for use in treating and/or preventing pain, migraine and/or symptoms of migraine in a subject, preferably a human patient.

The invention further relates, in particular, to a combination of (i) a ketone body and/or a ketogenic compound, and (ii) an analgesic, for use in treating and/or preventing headache, migraine and/or symptoms of migraine in a subject.

As illustrated in the appended Examples, the invention is, at least partly, based on the surprising finding that the combined administration of (i) β-hydroxybutyrate (βHB) and (ii) a simple analgesic (i.e. ibuprofen or acetylsalicylic acid), a triptan (i.e. frovatriptan) or melatonin ameliorated or eliminated acute headache in a migraine patient, whereas β-hydroxybutyrate alone, ibuprofen alone, acetylsalicylic acid alone, a triptan, or melatonin alone, were much less, and at best, only transiently effective. Moreover, in most instances, the inventive combination eliminated the headache within 2 hours after the intake. Astonishingly, when taken in the premonitory phase of an acute migraine attack, or at the onset of the headache, i.e. when the intensity of the headache was still low (i.e. about 0 to about 4 on a visual analogue scale ranging from 0 to 10), the combined treatment with (i) β-hydroxybutyrate (βHB) and (ii) ibuprofen, acetylsalicylic acid orfrovatriptan, completely eliminated the headache for at least 24 h in said patient, whereas the headache returned and/or got stronger within at least about 4-12 hours in cases β-hydroxybutyrate (βHB), ibuprofen, acetylsalicylic acid or frovatriptan was administered alone.

The inventors thus surprisingly found that a ketone body such as β-hydroxybutyrate (βHB) or precursors thereof, and an analgesic such as ibuprofen or acetylsalicylic acid can have a synergistic effect that is highly useful for the treatment and/or prevention of headaches, migraine and/or symptoms of migraine. Furthermore, the inventors found that the combination of a ketone body or ketogenic compound with other analgesics, e.g. triptans or melatonin, may be also advantageous for the treatment and/or prevention of headaches, migraine and/or symptoms of migraine compared to the individual components alone. The inventors further surprisingly found that a ketone body such as β-hydroxybutyrate (βHB) or a precursor thereof also functions as a rescue drug and can be used, in combination with an analgesic, e.g. a nonsteroidal anti-inflammatory drug such as ibuprofen or acetylsalicylic acid, a triptan or melatonin, for treating acute headache and/or an acute migraine attack.

Without being bound by theory, a synergistic effect of a ketone body and an analgesic such as a nonsteroidal anti-inflammatory drug, a triptan or melatonin may occur because the ketone bodies, e.g. βHB, may tackle the root cause of the migraine attack, i.e. oxidative stress and/or an energy deficit, but do not mitigate the effects of pain causing peptides. Thus, ketone bodies alone may be not very effective in eliminating already existing pain, but rather prevent the occurrence and/or further aggravation of the headache. In contrast, analgesics such as acetylsalicylic acid or ibuprofen only mitigate the pain, but do not stop the underlying migraine attack, so when their pain-killing effect ceases, the migraine it is still there, and the symptoms of the migraine attack, e.g. the headache, return.

Furthermore, also without bound by theory, ketone bodies or ketogenic compounds and analgesics such as acetylsalicylic acid, ibuprofen, triptans or melatonin have anti-inflammatory properties, whose mechanisms of action might work synergistically on the (neuro)inflammation that is part of migraine, both as a cause and as a consequence of the attack.

In addition, the antioxidant properties of certain analgesics, e.g. melatonin, may contribute to the synergy with a ketone body or ketogenic compound. Without being bound by theory, as part of the body's pain reaction pro-inflammatory molecules and free radicals are released. These can be mitigated with antioxidative substances, such as melatonin, which is a very potent antioxidant that works also inside of the mitochondria, as described herein. Ketone bodies are not only anti-inflammatory themselves, but as a potent alternative energy substrate that requires less oxygen to be converted to ATP as compared to glucose they are taking away a cause for the inflammatory response hence working in synergy with the antioxidative compound, e.g. melatonin.

In particular, herein and in the context of the invention, migraine or symptoms of migraine comprise pain, i.e., headache. In particular, said headache is unilateral, e.g. in at 25% of the migraine attacks, but it may be also two-sided. Typically, said headache is throbbing or pulsating. In particular, the headache may be of moderate to high intensity (e.g. about 5 to about 10 on a visual analogue scale ranging from 0 to 10).

Thus, the headache, as used herein, may resemble the headache associated with migraine or headache that occurs during an acute migraine attack, e.g., it may be unilateral, throbbing or pulsating, and/or of a moderate to high intensity (e.g. about 5 to about 10 on a visual analogue scale ranging from 0 to 10). For example, the headache, as used herein and in the context of the invention, may be associated with or caused by drug or alcohol consumption or abuse, and thus occur, for example, in the context of a hang-over. Furthermore, in the context of the present invention, the headache may be part of Cluster headache (a migraine-related neurological disorder characterized by recurrent severe one sided headaches (15 min to 3h in duration), typically around the eye and often accompanied by eye watering, nasal congestion, and/or swelling around the eye on the affected side). Furthermore, the headache may be tension-type headache (the most common typically mild or moderate headache type, characterized by dull pain, tightness, or pressure around the forehead, behind the eyes and around the head and neck), or headache in or caused by other conditions, such as after traumatic brain injury (a disruption in the normal function of the brain that can be caused by a blow, bump or jolt to the head or the head violently hitting an object or when an object pierces the skull and enters cerebral tissue). The headache may be also associated with fibromyalgia (a condition with high comorbidity to migraine, characterized by widespread musculoskeletal pain accompanied by fatigue, sleep, memory and mood issues).

As already indicated above, there is a great comorbidity between migraine and other disorders of pain such as fibromyalgia (Penn (2019), BMJ Open 9(4); Onder (2019), Neurol Res 41(10)), cluster headache (Vollesen (2018), J Headache Pain, 19(1)) and rheumatoid arthritis (Kim (2021), BMJ Open 11(6); Jacob (2021), J Clin Med 10(2)). For example, depending on the study, about 55-92% of the fibromyalgia patients also suffer from migraine. This suggests that the pain observed in migraine, i.e. headache, might have shared pathophysiological mechanisms and root causes as in other conditions of pain. However, in diseases such as fibromyalgia or rheumatoid arthritis, the pain is not limited to headache. It is therefore contemplated that the combinations or compositions of the present invention may be also suitable for treating other pain (in addition or instead of headache), e.g., pain associated with fibromyalgia or rheumatoid arthritis.

Therefore, the inventive combination, composition, food product, food supplement, nutritional aid, medical food or food for special medical purposes (FSMP) described herein may be also used for preventing and/or treating pain, e.g., pain that is associated with and/or caused by migraine, fibromyalgia, cluster headache and/or rheumatoid arthritis. Preferably herein and in the context of the invention, the pain comprises or is headache, preferably associated with and/or caused by migraine.

The migraine and/or symptoms thereof, as used herein, preferably comprise in addition to the headache at least one, preferably at least 2, 3, 4, 5, 6, 7, 8, 9, or 10, further symptom(s) selected from the group consisting of: an aura; nausea, sickness or vomiting; light, noise and/or smell sensitivity; balance disturbance or loss of balance; word finding difficulties; sensory and/or motor disturbances; allodynia; cognitive difficulties such as lightheadedness, brain fog, and/or difficulties to focus or concentrate; confusion; frequent yawing; changes in appetite and food craving such as binge eating; tiredness, fatigue or low energy; mood changes, e.g. from depression to euphoria; increased thirst and urination; neck stiffness; facial pain; head tension; sore eyes; changes in temperature sensation such as feeling too hot or too cold; diarrhea or constipation; sweating; irritability, dizziness or feeling faint.

More preferably, the migraine and/or symptoms thereof comprise headache and at least one further symptom selected from the group consisting of: an aura; nausea, sickness and/or vomiting; and light, noise and/or smell sensitivity.

To avoid any confusion, the symptoms of migraine and an acute migraine attack are the same. Furthermore, the terms “acute migraine attack”, “migraine attack” and “acute migraine” are used interchangeably herein.

The difference between the treatment of “migraine” and the treatment of an acute “migraine attack” is mainly that the treatment of migraine includes, inter alia, the prevention of migraine attacks (preventive component), whereas the treatment of an acute migraine attack focuses predominantly on the treatment of an already existing migraine attack (rescue component). Yet, the treatment of an acute migraine attack may also comprise the prevention of the recurrence of the migraine attack and/or symptoms thereof within about 1 or 2 days, i.e. within the same attack.

In particular, herein, and in the context of the invention, the subject is a vertebrate, preferably a mammal such as a human or a non-human animal, for example, interalia, a monkey, dog, cat, horse, cow, pig, sheep, camel, mouse, rat, guinea pig or hamster, most preferably a human. Furthermore, the subject may be preferably a female.

Preferably herein and in the context of the present invention, the subject is a patient which is in need of medical attention and/or intervention, herein simply called a “patient”. The subject may be a patient who is suffering from or who is susceptible to suffering from migraine, fibromyalgia, cluster headache and/or rheumatoid arthritis or pain associated with and/or caused by migraine, fibromyalgia, cluster headache and/or rheumatoid arthritis. In particular, the subject may be a patient who is suffering from or who is susceptible to suffering from headache, migraine, and/or symptoms of migraine.

Preferably, herein, the headache, migraine and/or symptoms of migraine is prevented and/or treated in a subject that has previously suffered from at least one migraine attack and/or is suffering from recurrent migraine attacks. Thus, the patient may be suffering from 1 to 31 migraine days per month, preferably at least about 3 or 5 days, more preferably at least about 10 days. In preferred embodiments, the patient is a metabolic migraineur, i.e. a patient, wherein more than 50% of its migraines are triggered by a metabolic event such as, inter alia, fasting, skipping a meal, or exercise. However, in some embodiments, i.e. in the context of non-therapeutic uses, the subject is not a patient who is in need of medical attention and/or intervention, but a healthy subject, i.e. subject who is not suffering from a pain disorder, e.g. migraine. Furthermore, herein and in the context of the present invention, the subject may suffer from depression, mania, mood swings, and/or a reduced libido.

In particular, as used herein and in the context of the present invention, the term “ketone body” includes β-hydroxybutyric acid, β-hydroxybutyrate, and/or a pharmaceutically acceptable β-hydroxybutyrate salt, which are collectively denoted herein as “βHB”, as well as, acetoacetic acid, acetoacetate, and/or a pharmaceutically acceptable acetoacetate salt, which are collectively denoted herein as “AcAc” (see, e.g. CAS No. 541-50-4).

Said βHB, as used herein, further includes any enantiomer, i.e. D-βHB and/or L-βHB, and/or a racemate (i.e. D/L-βHB) of beta-hydroxybutyric acid and/or its conjugate base beta-hydroxybutyrate. Beta-hydroxybutyric acid is also known as β-hydroxybutyric acid or 3-hydroxybutyric acid, and beta-hydroxybutyrate is also known as or 3-hydroxybutyrate or 3-hydroxybutyrate (CAS No. 300-85-6).

Most preferably herein, and in the context of the present invention, the βHB is D-βHB.

D-βHB, also known as (R)-βHB, as used herein, refers to a specific enantiomer of βHB. Said enantiomer can be produced in the liver by humans. Furthermore, D-βHB or a precursor of D-βHB was shown to increase the brain metabolic efficiency and regulate the transcriptional and the epigenetic state of cells; WO2010021766A1 and Shimazu et al., Science. 2013 Jan. 11; 339(6116):211-4.

The formulae of β-hydroxybutyric acid (left), D-β-hydroxybutyric acid (middle) and D-β-hydroxybutyrate (right) are depicted in the following:

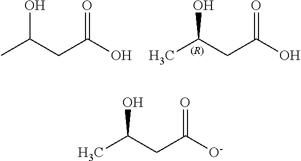

The formula of acetoacetate is depicted in the following:

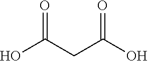

βHB and AcAc are the major types of ketone bodies that are produced by humans in response to fasting or a ketogenic diet, and thus may also refer to “endogenous ketone bodies” which may be measured, e.g., in the blood of a subject as described herein. In particular, βHB and AcAc are produced by the liver from fatty acids released from adipose tissue in times of starvation, fasting, glucose and/or carbohydrate deprivation and/or prolonged intense exercise. They can be used as an alternative energy substrate to glucose by most tissues of the body, most notably the brain, which cannot metabolize any other energy substrate apart from glucose, lactate and ketone bodies.

However, βHB and AcAc may also refer to “exogenous ketone bodies” when they are administered to a subject, as described herein and in the context of the invention, although they may be chemically identical to the “endogenous ketone bodies”.

The terms “ketone body”, “ketogenic compound”, “βHB”, and “ketone ester”, as used herein and in the context of the present invention, do, in particular, not include poly-3-hydroxybutyrate, poly-D-(−)-3-hydroxybutryic acid, a co-polymer of poly-3-hydroxybutyrate such as poly(β-malic acid)-b-poly(β-hydroxybutyrate) and/or other polyesters that comprise or consist of at least 10000, 5000, 1000, 500, 200, 100, 50, 20, or 10 units, e.g. at least 100 units, of monomeric βHB and that are not effectively metabolized into monomeric βHB in the body and/or that are used for the production of plastic such as biodegradable plastic, e.g. poly(3-hydroxybutyrate-co-3-hydroxyvalerate), or poly(3-hydroxybutyrate-co-4-hydroxybutyrate). In particular, the inventive composition provided herein may be not comprised in volume and/or weight of more than about 90%, 70%, 50%, 30%, 10%, 1%, or more than about 0% poly-3-hydroxybutyrate, poly-D-(−)-3-hydroxybutryic acid, a co-polymer of poly-3-hydroxybutyrate such as poly(β-malic acid)-b-poly(β-hydroxybutyrate) and/or other non-ketogenic polyesters.

In particular, as used herein and in the context of the present invention, the term “ketogenic compound” refers to a precursor of βHB and/or AcAc. In particular, a ketogenic compound, as used herein, is a specific chemical compound or a defined mix of chemical compounds. In particular, the term “ketogenic compound”, as used herein, does not refer to a naturally occurring composition or food to which no specific chemical compound has been added.

A precursor of an endogenous ketone body, i.e. βHB and/or AcAc, produces βHB and/or AcAc upon administration to a subject or during preparation of said precursor for administration to a subject. Preferably herein, the precursor of βHB and/or AcAc is a metabolic precursor of βHB and/or AcAc. In particular, a metabolic precursor is metabolized in the body of a subject as described herein into βHB and/or AcAc. For example, a metabolic precursor, when administered to a human or animal body, is metabolized, e.g. in the liver, to produce D-βHB and AcAc, preferably in a physiological ratio.

Preferably herein and in the context of the invention, the ketone body and/or ketogenic compound comprises a ketone body, preferably βHB, more preferably D-βHB.

As used herein, the βHB may include D-βHB and/or L-βHB, the racemic DL-βHB or exclusively the isomer D-βHB. Preferably, the βHB contains at least about 50%, 60%, 70%, 80%, 90%, 95%, 99%, 99.9%, 99.99% or 100%, preferably at least about 90%, more preferably at least about 99%, most preferably about 100% D-βHB, and/or less than about 50%, 40%, 30%, 20%, 10%, 5%, 1%, 0.1%, 0.01% or 0%, preferably less than about 10%, more preferably less than about 1%, most preferably about 0% L-βHB. In some embodiments, the βHB contains at least about 50%, 60%, 70%, 80%, 90%, 95%, 99%, 99.9%, 99.99% or 100% L-βHB. However, as mentioned above, herein and in the context of the invention, βHB preferably refers to D-βHB.

In a preferred embodiment, the ketone body comprises a pharmaceutically acceptable β-hydroxybutyrate salt.

A pharmaceutically acceptable salt, as used herein, may be selected from the group consisting of: a potassium salt, a sodium salt, a calcium salt, a magnesium salt, an arginine salt, a zinc salt, a barium salt, an iron salt, a copper salt, a manganese salt, molybdenum salt, a lithium salt, a phosphor salt, a sulfur salt, an iodine salt, a selenium salt, a chromium salt, a cobalt salt, a lysine salt, a leucine salt, a histidine salt, an ornithine salt, a creatine salt, an agmatine salt, a citrulline salt, a methyl glucamine salt and a carnitine salt, or a combination of at least two of said salts.

Preferably, the pharmaceutically acceptable salt comprises at least 1, 2, 3 or 4, preferably all, salts selected from the group consisting of: a calcium salt, a sodium salt, a potassium salt, and a magnesium salt. For example, the pharmaceutically acceptable salt may be a blend of sodium, calcium, potassium, magnesium mineral salts at a mixing ratio of about 1:2:1:2, about 1:1:1:2 or about 1:1:1:1.

It is preferred to use a combination of several salts. In particular, by increasing the number of different salts, the total tolerated dose may be increased.

Furthermore, the ketone body, as used herein and in the context of the present invention, may comprise β-hydroxybutyric acid. Thus, the ketone body may comprise a pharmaceutically acceptable β-hydroxybutyrate salt and β-hydroxybutyric acid. For example, the ketone body and/or ketogenic compound may comprise or consist of an (1:1) mix of D-β-hydroxybutyric acid and a pharmaceutically acceptable salt mix of D-β-hydroxybutyrate, as described herein, and optionally further a desiccant as described herein.

Preferably herein, the ketone body comprises D-β-hydroxybutyrate and/or D-β-hydroxybutyric acid, and/or said ketogenic compound is metabolized into D-β-hydroxybutyrate and/or D-β-hydroxybutyric acid. It is also preferred herein and in the context of the invention that the ketone body does not comprise L-β-hydroxybutyrate and/or L-β-hydroxybutyric acid, and/or said ketogenic compound is not metabolized into L-β-hydroxybutyrate and/or L-β-hydroxybutyric acid.

Herein and in the context of the present invention, the ketogenic compound may comprise a metabolic precursor of βHB and/or AcAc selected from the group consisting of: 1,3-butanediol, triacetin, a fatty acid such as a medium chain fatty acid, or a compound comprising a fatty acid such as a medium chain fatty acid, e.g. a medium chain triglyceride, or a ketogenic amino acid such as leucine or lysine; or a pharmaceutically acceptable salt of said metabolic precursor. In particular, 1,3-butanediol refers to CAS No. 107 88 0, and triacetin refers to CAS No. 102-76-1.

In particular herein and in the context of the present invention, the term “fatty acid” includes short chain fatty acids, medium chain fatty acids, and long chain fatty acids as described herein, e.g. acids with aliphatic tails of 4 to 21 carbon atoms, i.e. 4, 5, 6, 7, 8, 9, 10, 11, 12, 13, 14, 15, 16, 17, 18, 19, 20, or 21 carbon atoms, e.g. 4, 5, 6, 7 or 8 carbon atoms, preferably 6 or 8, carbon atoms, more preferably 8 carbon atoms. Preferably herein, said chain (aliphatic tail) is saturated.

In particular herein in the context of the present invention, the terms “medium chain fatty acid”, “middle chain fatty acid” or “MCFA”, refer to an aliphatic monocarboxylic acid comprising a chain of 6, 7, 8, 9, 10, 11 or 12 carbon atoms, preferably 6 or 8 carbon atoms, more preferably 8 carbon atoms. Thus, the MCFA include preferably caproic acid, caprylic acid, capric acid and lauric acid, preferably caprylic acid or capric acid, more preferably caprylic acid. However, in the context of the present invention, also other fatty acids can be used in place of a medium chain fatty acid, e.g. long chain fatty acids with aliphatic tails of 13 to 21 carbons, or short chain fatty acids with 4 or 5 carbon atoms i.e., as long as they can be metabolized into βHB and/or AcAc. In particular, short chain fatty acids with 4 or 5 carbon atoms are very ketogenic and thus can be well used instead of medium chain fatty acids herein and in the context of the present invention.

The term “medium chain triglyceride”, “middle chain triglyceride” or “MCT”, as used herein, refer to an ester that is formed by the union of glycerol (see, e.g., CAS No. 56-81-5) and three MCFA. Furthermore, herein and in the context of the present invention, a MCT may be replaced by an ester that is formed by the union of glycerol and three short-, middle- or long-chain fatty acids, or a combination thereof.

As used herein, the term “ketogenic amino acid” refers to an amino acid that can be degraded to Acetyl-CoA, a precursor of endogenous ketone bodies. Leucine and lysine are ketogenic amino acids that are exclusively ketogenic. Isoleucine, phenylalanine, tryptophan and tyrosine are ketogenic amino acids that are also glucogenic. Thus, the ketogenic amino acid may comprise, in particular, leucine lysine, isoleucine, phenylalanine, tryptophan and/or tyrosine.

Furthermore, the ketogenic compound may comprise a compound comprising an acetoacetyl- and/or 3-hydroxybutyrate moiety.

An ester is a chemical compound derived from an acid in which at least one —OH hydroxyl group is replaced by an —O— alkyl (alkoxy) group as in the substitution reaction of a carboxylic acid and an alcohol. Thus, herein, an ester formed by the union of two compounds means that these two compounds are esterified, in particular via an acid group of one compound and an alcohol group of the other compound.

Preferably herein and in the context of the present invention, the ketogenic compound comprises a ketone ester, or a pharmaceutically acceptable salt thereof, preferably a ketone ester.

The term “ketone ester” herein, refers to any ester that is a precursor, preferably a metabolic precursor, of βHB and/or AcAc, as described herein. Thus, a ketone ester may comprise, in particular, a 3-hydroxybutyrate (βHB) moiety, an acetoacetyl (AcAc) moiety, and/or at least one moiety that itself is a precursor of βHB and/or AcAc such as a 1,3-butanediol moiety or a middle chain fatty acid moiety.

Thus, in the context of the present invention, the ketone ester may comprise an ester of βHB and/or AcAc.

Thus, the ketone ester may comprise an ester formed by the union of βHB with βHB, βHB with AcAc, or AcAc with AcAc, e.g., 3-hydroxybutyl-(R)-3-hydroxybutyrate.

Furthermore, the ketone ester may comprise an ester formed by the union of βHB or AcAc with a monohydric, dihydric or trihydric alcohol. For example, the ketone ester may comprise an ester formed by the union of βHB with 1,3-butanediol or glycerol, wherein the 1,3-butanediol or glycerol may be further esterified with a fatty acid, e.g., a medium chain fatty acid. Thus, the ketone ester may comprise, for example, an ester formed by the union of βHB or AcAc with 1,3-butanediol, for example, an (R)-3-hydroxybutyrate-R-1,3-butanediol monoester. In a further example, the ketone ester may comprise an ester formed by the union of βHB or AcAc with glycerol, for example, glyceryl tris(3-hydroxybutyrate. The glycerol may be further esterified with at least one fatty acid, e.g., a medium chain fatty acid.

The ketone ester may also comprise an ester formed by the union of βHB or AcAc (preferably βHB) with a fatty acid, e.g. a medium chain fatty acid.

Furthermore, the ketone ester may comprise an ester formed by the union of 1,3-butanediol with at least one fatty acid, e.g. a medium chain fatty acid, for example, 1,3-butanediol esterified with two caproic acids.

Preferably herein, the ketone ester is an ester of D-βHB and/or the ketone ester is metabolized in the body of a subject into D-βHB and/or acetoacetate, preferably at least D-βHB.

Furthermore, the ketogenic compound may comprise an amide of βHB and/or AcAc, or a pharmaceutically acceptable salt thereof.

Furthermore, the ketogenic compound may comprise a compound selected from the group consisting of: D-beta-hydroxybutyrate-D-1,3-butanediol; (3R)-hydroxybutyl-(3R)-hydroxybutyrate; acetoacetyl-1,3-butanediol; acetoacetyl-R-3-hydroxybutyrate; acetoacetylglycerol; 3-hydroxybutyl 3-hydroxybutanoate; (3-hydroxy-1-methyl-propyl) 3-hydroxybutanoate; 3-(3-hydroxybutanoyloxy)butyl 3-hydroxybutanoate; 3-(3-hydroxybutanoyloxy)butanoic acid; 3-hydroxybutyl 3-oxobutanoate; 3-hydroxy-1-methyl-propyl) 3-oxobutanoate; 3-(3-oxobutanoyloxy)butyl 3-oxobutanoate; 3-(3-oxobutanoyloxy)butanoic acid; 2,3-dihydroxypropyl 3-oxobutanoate; [2-hydroxy-1-(hydroxymethyl)ethyl]3-oxobutanoate; [2-hydroxy-3-(3-oxobutanoyloxy)propyl]3-oxobutanoate; [3-hydroxy-2-(3-oxobutanoyloxy)propyl]3-oxobutanoate; 2,3-bis(3-oxobutanoyloxy)propyl 3-oxobutanoate; and any one of the formulae

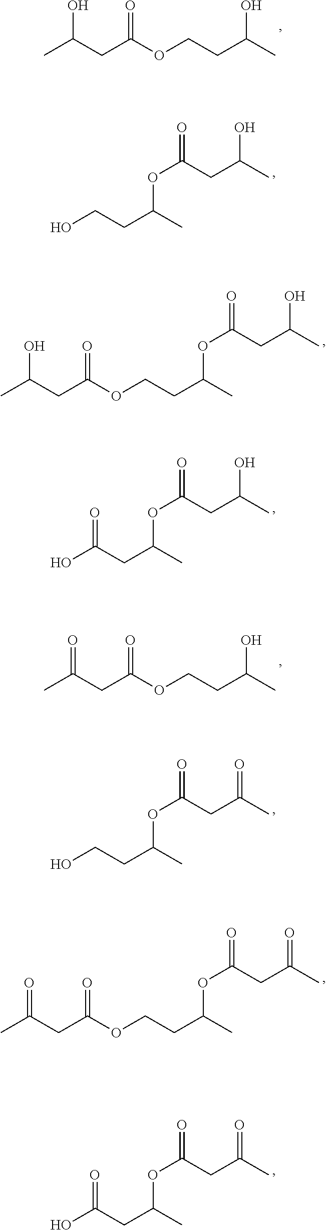

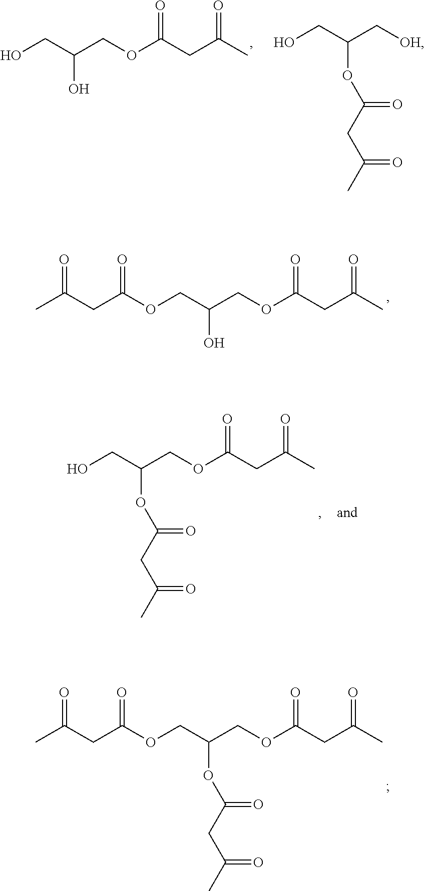

and a pharmaceutically acceptable salt thereof.

In some embodiments of the invention, the ketone body and/or ketogenic compound does not comprise B-hydroxybutyrate-1,3-butanediol monoester, glyceryl-tris-3-hydroxybutyrate, R-3-hydroxy-butyrate-R-1,3-butanediol monoester and/or D-B-hydroxybutyrate-(R)-1,3-butanediol.

In some embodiments of the invention, the ketone body and/or ketogenic compound does not comprise a medium chain triglyceride.

In some embodiments of the invention, the ketone body and/or ketogenic compound does not comprise B-hydroxybutyrate-1,3-butanediol monoester, glyceryl-tris-3-hydroxybutyrate, R-3-hydroxy-butyrate-R-1,3-butanediol monoester, D-B-hydroxybutyrate-(R)-1,3-butanediol and/or a medium chain triglyceride.

An analgesic, as used herein and in the context of the invention, may be also termed a “painkiller”, and is used to achieve analgesia or relief from pain, in particular, without completely eliminating sensation. Furthermore, the analgesic, e.g. a nonsteroidal anti-inflammatory drug such as ibuprofen or acetylsalicylic acid, or melatonin, may be anti-inflammatory. Furthermore, an analgesic such as melatonin may have antioxidant properties.

In particular, the analgesic may comprise a nonsteroidal anti-inflammatory drug, an inhibitor of cyclooxygenase, acetaminophen (Paracetamol), a triptan, melatonin, an opioid and/or a cannabioid. Preferably said inhibitor of cyclooxygenase inhibits at least COX2, and it may inhibit exclusively COX2 but not COX1 or both, COX2 and COX2.

There may be also an overlap between these classes, as, for example, some nonsteroidal anti-inflammatory drugs (NSAIDs) may be also inhibitors of cyclooxygenase. Preferably herein, the analgesic comprises or consists of simple analgesics. In particular, simple analgesics, as used herein, include nonsteroidal anti-inflammatory drugs such as acetylsalicylic acid and ibuprofen, inhibitors of cyclooxygenase, and/or acetaminophen.

Preferably herein, the analgesic, e.g. the simple analgesic, comprises at least one nonsteroidal anti-inflammatory drug. In particular, the nonsteroidal anti-inflammatory drug(s) may be selected from the group consisting of the following (i) to (vii):
(i) a salicylate such as acetylsalicylic acid (e.g. Aspirin), diflunisal (e.g. Dolobid), salicylic acid or a salt thereof, or salsalate (e.g Disalcid);
(ii) a propionic acid derivative such as ibuprofen, dexibuprofen, naproxen, fenoprofen, ketoprofen, dexketoprofen, flurbiprofen, oxaprozin, Ioxoprofen, pelubiprofen, or zaltoprofen;
(iii) an acetic acid derivative such as diclofenac, indomethacin, tolmetin, sulindac, etodolac, ketorolac, aceclofenac, bromfenac, or nabumetone;
(iv) an enolic acid (e.g. oxicam) derivative such as piroxicam, meloxicam, tenoxicam, droxicam, lornoxicam, or phenylbutazone (e.g. Bute);
(v) an anthranilic acid derivative (fenamate) such as mefenamic acid, meclofenamic acid, flufenamic acid, or tolfenamic acid;
(vi) a selective COX-2 inhibitor (coxib) such as celecoxib, parecoxib, or etoricoxib; and
(vii) clonixin, licofelone or H-harpagide.

However, nonsteroidal anti-inflammatory drugs that are suitable for the present invention are not limited by the above group (i) to (vii) of nonsteroidal anti-inflammatory drugs.

Preferably, the analgesic, i.e. the nonsteroidal anti-inflammatory drug, comprises a salicylate and/or a propionic acid derivative such as ibuprofen, dexibuprofen, dexketoprofen or naproxen. Most preferably herein the analgesic, i.e. the nonsteroidal anti-inflammatory drug, comprises acetylsalicylic acid and/or ibuprofen. Ibuprofen is also called isobutylphenylpropionic acid.

Thus, in a particularly preferred embodiment, the ketone body or ketogenic compound comprises a pharmaceutically acceptable β-hydroxybutyrate salt, and said analgesic comprises acetylsalicylic acid or ibuprofen.

Triptans are serotonin receptor agonists and have several different possible mechanisms of action, i.e. vasoconstriction of pain-sensitive intracranial vessels by acting on vascular smooth muscle; and inhibition of the release of vasoactive and/or proinflammatory neuropeptides, such as calcitonin gene-related peptide (CGRP) and substance P, e.g. from trigeminal afferents. For example, CGRP is a pro-inflammatory neuropeptide involved in pain transmission, in particular, in migraine. Via their inhibition of CGRP release triptans can be classified as a pain killer, i.e. as an analgesic in the context of the present invention.

Suitable triptans, i.e. for the use of the treatment of an acute migraine attack or cluster headache, include, inter alia, sumatriptan, zolmitriptan, naratriptan, rizatriptan, almotriptan, eletriptan, frovatriptan, donitriptan and avitriptan. In some embodiments, frovatriptan is used in combination with a ketone body and/or ketogenic compound, e.g. βHB, as described herein.

Thus, in some embodiments, the analgesic or the inventive composition provided herein comprises a triptan, e.g. frovatriptan.

Hence, in some embodiments, the ketone body or ketogenic compound comprises a pharmaceutically acceptable β-hydroxybutyrate salt, and said analgesic comprises a triptan.

In some embodiments, the ketone body or ketogenic may be used in combination with a simple analgesic such as acetylsalicylic acid or ibuprofen, and a triptan.

Melatonin is a hormone primarily released by the pineal gland at night that is associated with control of the sleep-wake cycle. Melatonin is also called N-[2-(5-Methoxyindol-3-yl)ethyl]acetamid or N-Acetyl-5-Methoxytryptamin; i.e. CAS-Nr. 73-31-4. Melatonin is often used for the short-term treatment of insomnia, such as from jet lag or shift work, and is typically taken orally.

Recent evidence indicates that melatonin has analgesic properties, and may be used, e.g. for the treatment of headache such as, inter alia, cluster headaches (Xie (2020), J Pain Res. 13; Chen (2016), Exp Ther Med. 12(4); Peres (2006), Expert Opinion on Investigational Drugs. 15 (4); Evers (2005), Practical Neurology. 5 (3)).

It is further well known that melatonin is an antioxidant (Tan (2015), Molecules 20(10); Chitimus (2020), Biomolecules 10(9); Hardeland (2005), Nutr Metab (Lond) 2; Reiter (2016), Journal of Pineal Research 61).

As described in Hardeland (2005), Nutr Metab (Lond) 2, due to its amphiphilicity, melatonin can enter any body fluid, cell or cell compartment. Apart from direct radical scavenging, melatonin plays a role in upregulation of antioxidant and downregulation of prooxidant enzymes. Melatonin has been shown to potentiate effects of other antioxidants, such as ascorbate and Trolox.

Furthermore, melatonin acts inside mitochondria. It has been suggested that melatonin should be classified as a mitochondria-targeted antioxidant. Furthermore, it has been reported that melatonin chelates transition metals, which are involved in the Fenton/Haber-Weiss reactions (Reiter (2016), Journal of Pineal Research 61).

Therefore, melatonin reduces, inter alia, the formation of the toxic hydroxyl radical resulting in the reduction of oxidative stress.

In particular, herein and in the context of the present invention, the antioxidant reduces oxidative stress in biological tissue and/or cells. Therefore, the antioxidant may be a biological antioxidant.

Furthermore, the antioxidant may be (i) a free radical scavenger and/or capable of directly detoxifying reactive oxygen and/or reactive nitrogen species, (ii) stimulate antioxidant enzymes such as superoxide dismutase, glutathione peroxidase, glutathione reductase and/or catalase, and/or suppress the activity of pro-oxidant enzymes such as nitric oxide synthase, and/or (iii) chelate transition metals that are involved, e.g., in Fenton-Haber-Weiss reactions.

Furthermore, the antioxidant may be a mitochondria-targeted antioxidant, i.e., the antioxidant may function inside mitochondria and/or be capable of entering mitochondria.

Furthermore, the antioxidant may be lipophilic or amphiphilic, preferably amphiphilic. Advantageously, amphiphilic antioxidants such as melatonin, alpha-lipoic acid or astaxanthin, protect both, the aqueous and lipid part of cells.

There are various different substances that have an at least equal or greater antioxidant capacity compared to melatonin (Rodriguez-Naranjo (2012), Journal of Food Composition and Analysis, Vol. 28, Issue 1).

For example, resveratrol is a known as a strong antioxidant (Mu6 oz (2020), Arch Med 16(4)). Further suitable antioxidants are ascorbic acid/Vitamin C and Vitamin E (Naziroylu (2010), Cell Biochemistry & Function, Vol. 28, Issue 4; Vitamin C (2017), published by InTech, Rijeka, Croatia - Pehlivan, page 23; Machlin (1986), Advances in Free Radical Biology & Medicine, Vol. 2, Issue 2), alpha-lipoic acid (Packer (1995), Free Radical Biology & Medicine, Vol. 19, No. 2); Tibullo (2017), Inflamm Res 66(11)), Coenzyme Q10 (Saini (2011), J Pharm Bioallied Sci. 3(3); Lee (2012), Nutrition, Vol. 28, Issue 3), and astaxanthin.

It is further known that melatonin is an indoleamine and that 5-hydroxy-indoles have high antioxidant activity (Rodriguez-Naranjo (2012), Journal of Food Composition and Analysis, Vol. 28, Issue 1).

Therefore, herein and in context of the present invention, the antioxidant may comprise an indolamine such as melatonin, an indol such as an 5-hydroxy-indole or indole-3-acetic acid, a polyphenol such as resveratrol, alpha-lipoic acid, astaxanthin, ascorbic acid, Vitamin E, kaempferol, and/or coenzyme Q.

Furthermore, the antioxidant may instead of or in addition to melatonin a melatonin receptor agonist such as agomelatine and/or tasimelteon.

Therefore, herein and in the context of the present invention, agomelatine or tasimelteon may be used instead of melatonin. However, herein and in the context of the present invention, melatonin is preferred over agomelatine or tasimelteon.

Most preferably herein and in context of the present invention, the antioxidant comprises or is melatonin.

The combination or composition of the present inventive may comprise a ketone body or ketogenic compound (e.g. β-hydroxybutyrate), an analgesic (e.g. ibuprofen or acetylsalicylic acid) and an antioxidant (e.g. melatonin) as described herein. However, the analgesic and the antioxidant may be also the same compound, e.g. melatonin.

Thus, in some embodiments, the analgesic and/or antioxidant or the inventive composition provided herein comprises melatonin.

Hence, in some embodiments, the ketone body or ketogenic compound comprises a pharmaceutically acceptable β-hydroxybutyrate salt, and said analgesic and/or antioxidant comprises melatonin. In some embodiments, the ketone body or ketogenic may be used in combination with a simple analgesic such as acetylsalicylic acid or ibuprofen, and melatonin. In further embodiments, the ketone body or ketogenic may be used in combination with a triptan and melatonin.

Cannabinoids that are suitable for use as analgesic in the present invention include, inter alia, cannabidiol (CBD), cannabimimetics such as aminoalkylindoles, 1,5-diarylpyrazoles, quinolines, and arylsulfonamides as well as eicosanoids (which are related to endocannabinoids), tetrahydrocannabinol (THC), synthetic cannabinoids that mimic the effects of THC, and synthetic cannabinoids that mimic the effects of CBD. Preferably, herein and in the context of the present invention the cannabinoid comprises or consists of cannabidiol.

Thus, in some embodiments, the analgesic or the inventive composition provided herein comprises a cannabinoid, e.g. cannabidiol.

Hence, in some embodiments, the ketone body or ketogenic compound comprises a pharmaceutically acceptable β-hydroxybutyrate salt, and said analgesic comprises a cannabinoid.

In some embodiments, the ketone body or ketogenic may be used in combination with a simple analgesic such as acetylsalicylic acid or ibuprofen, and a cannabinoid.

In some embodiments, the ketone body or ketogenic may be used in combination with a simple analgesic such as acetylsalicylic acid or ibuprofen, a triptan, melatonin, a cannabinoid or any combination of said analgesics.

Opioids that are suitable for use as analgesic in the present invention include, inter alia, hydrocodone or dihydrocodeinone (e.g., interalia, Vicodin®, Norco@, Zohydro®), oxycodone (e.g. OxyContin® or Percocet®), oxymorphone (e.g. Opana®), morphine (e.g. Kadian®, Avinza®, Duramorph®, MS Contin®), codeine, fentanyl (e.g Actiq®, Duragesic®,or Sublimaze®), Hydromorphone (e.g. Dilaudid®), Meperidine (e.g. Demerol®), and Methadone (e.g. Dolophine®, Methadose®).

Furthermore, the invention relates to a composition comprising (i) a ketone body and/or a ketogenic compound, and (ii) an analgesic and/or an antioxidant, as described herein.

In some embodiments, the composition comprises (i) a ketone body and/or a ketogenic compound, and (ii) melatonin and/or a melatonin receptor agonist such as melatonin, agomelatine and/or tasimelteon, preferably melatonin.

The composition of the invention is, in particular, suitable for alleviating pain as described herein. Said pain may be associated with and/or caused by migraine, fibromyalgia, cluster headache and/or rheumatoid arthritis, preferably migraine.

Preferably said pain comprises or is headache, e.g. headache associated and/or caused by migraine.

Therefore, the inventive composition provided herein is, in particular, suitable for treating and/or preventing headache, migraine and/or symptoms of migraine in a subject, as described herein.

Thus, the inventive composition provided herein may be used for treating and/or preventing migraine, cluster headache, fibromyalgia, epilepsy, traumatic brain injury, depression, anxiety disorders, multiple sclerosis, Parkinson's disease, Alzheimer's disease, chronic fatigue syndrome, and/or chronic pain conditions. For example, the inventive composition may be used for treating and/or preventing migraine, cluster headache, fibromyalgia and/or rheumatoid arthritis, preferably migraine, as described herein.

Furthermore, the inventive composition provided herein may be used for treating and/or preventing headache, migraine and/or symptoms of migraine in a subject, as described herein.

The inventive composition may be comprised in a food product or food supplement. Thus, the invention also relates to a food product, food supplement or nutritional aid comprising the inventive composition provided herein, i.e. (i) a ketone body and/or a ketogenic compound, and (ii) an analgesic and/or an antioxidant, as described herein.

In some embodiments of the inventive composition, food product, food supplement or nutritional aid, the analgesic comprises or consists of one or more cannabinoids. For example, the cannabinoids may comprise or consist of CBD.

In some embodiments of the inventive composition, food product, food supplement or nutritional aid, the analgesic and/or antioxidant comprises or consists of melatonin.

Preferably, the composition further comprises β-hydroxybutyric acid, β-hydroxybutyrate, and/or a pharmaceutically acceptable β-hydroxybutyrate salt.

In some embodiments of the inventive composition, food product, food supplement or nutritional aid, the analgesic and/or antioxidant comprises or consists of melatonin and/or at least one cannabinoid, preferably CBD.

In some embodiments, the food product, food supplement or nutritional aid comprises (i) a ketone body and/or a ketogenic compound, and (ii) melatonin and/or a melatonin receptor agonist such as agomelatine and/or tasimelteon, preferably melatonin.

In a preferred embodiment, the food product, food supplement or nutritional aid comprises (i) β-hydroxybutyric acid, β-hydroxybutyrate, and/or a pharmaceutically acceptable β-hydroxybutyrate salt, preferably a pharmaceutically acceptable β-hydroxybutyrate salt, and (ii) melatonin.

In particular, the inventive food product, food supplement or nutritional aid may promote the well-being of a subject, e.g., by alleviating pain, i.e. headache, neck tension, cognitive difficulties, sleep disturbances, or other migraine or headache associated symptoms and/or promoting and supporting brain health and/or cognitive function, such as reducing brain hyperexcitability (a common phenomenon in epilepsy and migraine and other brain health issues), which may reduce the frequency and/or intensity of migraine attacks or related syndromes and conditions as a consequence.

In some embodiments, the inventive composition, i.e. the inventive food product, food supplement or nutritional aid, more specifically a food product, food supplement or nutritional aid comprising (i) a ketone body and/or ketogenic compound as described herein, and (ii) a cannabinoid (e.g CBD) and/or melatonin, may be used for solely non-therapeutic purposes, e.g. for promoting and/or supporting cognitive performance, self-confidence, restful sleep, and/or mental well-being, in particular in a subject that is not in need of medical attention or intervention. Preferably, in the context of non-therapeutic uses, the analgesic and/or antioxidant comprises or consists of one or more cannabinoids and/or melatonin. For example, the cannabinoids may comprise or consist of CBD.

The terms “food” and “food product” are used interchangeably herein and refer to any substance consumed to provide nutritional support for an organism. Food may be of biological origin, and contain essential nutrients, such as carbohydrates, fats, proteins, vitamins, or minerals and/or consist essentially of an aqueous solution. Food is ingested by a subject and assimilated by the subject's cells to provide energy, maintain life, or stimulate growth. A food product can be taken in by a subject or administered to a subject. Preferably, a food product is eaten or drunk.

A food supplement, as used herein, refers to a concentrated source of nutrients with a nutritional and/or physiological effect. In the present invention, the main nutrient comprised in a food supplement may be the ketone body and/or ketogenic compound described herein. A food supplement may further comprise other nutrients, in particular vitamins or minerals. A food supplement can be taken in by a subject or administered to a subject. Preferably, a food supplement is eaten or drunk.

The food product may further comprise a further edible and/or drinkable material.

Furthermore, the inventive composition may be formulated as medical food and/or food for special medical purposes. A medical food and/or food for special medical purposes may be also considered a food product, food supplement or nutritional aid as described herein.

Therefore, the invention further relates to a medical food or food for special medical purposes (FSMP) comprising (i) a ketone body and/or a ketogenic compound, and (ii) an analgesic and/or an antioxidant, as described herein.

In some embodiments, the medical food or food for special medical purposes (FSMP) comprises (i) a ketone body and/or a ketogenic compound, and (ii) melatonin and/or a melatonin receptor agonist such as agomelatine and/or tasimelteon, preferably melatonin.

In one embodiment, the medical food or food for special medical purposes (FSMP) comprises (i) β-hydroxybutyric acid, β-hydroxybutyrate, and/or a pharmaceutically acceptable β-hydroxybutyrate salt, preferably a pharmaceutically acceptable β-hydroxybutyrate salt, and (ii) melatonin.

Furthermore, a food for special medical purposes, as used herein, refers to food products for the dietary management (under medical supervision), of individuals who suffer from certain diseases, disorders or medical conditions. These foods are intended for the exclusive or partial feeding of people whose nutritional requirements cannot be met by normal foods.

Furthermore, a medical food, as used herein, refers to a food which is formulated to be consumed or administered enterally under the supervision of a physician and which is intended for the specific dietary management of a disease or condition for which distinctive nutritional requirements, based on recognized scientific principles, are established by medical evaluation.

A food for special medical purposed and/or a medical food relates, in particular, to the therapeutic uses of the invention.

Preferably herein, the inventive composition is a pharmaceutical composition and/or a medicament. Thus, the inventive composition provided herein may be used for treating a disease.

Furthermore, the invention relates to a method for treating and/or preventing a disease in a subject comprising administering to said subject an effective amount of the inventive combination or composition provided herein.

The terms “disorder”, “disease” and “medical condition” are used herein interchangeably and may refer to a pathophysiological response to external or internal factors, a disruption of normal or regular functions in the body or a part of the body and/or an abnormal state of health that interferes with the usual activities or feeling of wellbeing.

As used herein, “treatment” (and grammatical variations thereof such as “treat” or “treating”) refers to clinical intervention in an attempt to alter the natural course of the individual being treated. Desirable effects of treatment include, but are not limited to, prophylaxis, preventing occurrence or recurrence of disease or symptoms associated with disease, alleviation of symptoms, diminishment of any direct or indirect pathological consequences of the disease, decreasing the rate of disease progression, amelioration or palliation of the disease state, improved prognosis and cure.

In particular, as used herein, the term “treatment” and grammatical variations thereof refers to the alleviation or elimination of symptoms, the amelioration of the disease state, and/or preventing the recurrence of disease or symptoms associated with disease. The term “prevention” (and grammatical variations thereof such as “prevent” or “preventing”) refers in, particular, to prophylaxis, and/or preventing occurrence or recurrence of disease or symptoms associated with disease. Furthermore, the term “amelioration” refers, in particular, to a decrease in the intensity, duration and/or frequency of the symptoms of disease, and may also include the prevention of the aggravation of the symptoms and/or the prevention of the recurrence of aggravated symptoms.

Herein and in the context of the present invention, the disease may be a disorder of pain such as migraine, cluster headache, fibromyalgia and/or rheumatoid arthritis.

In other words, the disease may be associated with pain, preferably headache, as described herein.

In particular, the treatment may comprise alleviating or treating pain, e.g., pain associated with and/or caused by migraine, fibromyalgia, cluster headache and/or rheumatoid arthritis. Preferably said pain comprises or is headache.

Furthermore, the disease may be treatable by a ketone body and/or ketogenic compound as described, e.g. in WO2020229364. Furthermore, herein and in the context of the invention, i.e. in context of the inventive composition for use in treating a disease, the disease may be a neurological and/or brain disorder such as, inter alia, migraine, cluster headache, fibromyalgia, epilepsy, traumatic brain injury, depression, an anxiety disorder (i.e. when then analgesic comprises a cannabinoid such as CBD), multiple sclerosis (MS), a neurodegenerative disorder such as Parkinson's disease or Alzheimer's disease, chronic fatigue syndrome (CFS), or a chronic pain condition, preferably migraine as described herein.

More preferably herein, the inventive composition is used for treating and/or preventing headache, migraine and/or symptoms of migraine in a subject as described herein, i.e. in the context of the combination of (i) a ketone body and/or a ketogenic compound, and (ii) an analgesic, for use in treating and/or preventing headache, migraine and/or symptoms of migraine in a subject.

The inventive combination or composition provided herein is particularly useful for treating and/or preventing pain (e.g. headache), migraine and/or symptoms of migraine that are refractory to treatment with acetylsalicylic acid or ibuprofen as single active ingredient. Refractory to treatment means that the pain (e.g. headache), migraine and/or symptoms of migraine are not ameliorated by the treatment or that they are ameliorated by the treatment only for a short time, e.g. for at most about 6, 8, 10 or 12 h, e.g. about 8 hours, before the headache, migraine and/or symptoms of migraine return.

Furthermore, the pain (e.g. headache), migraine and/or symptoms of migraine may be refractory to treatment with a triptan, e.g. frovatriptan, as single active ingredient. Migraine is characterized by the occurrence of migraine attacks. Thus, the treatment of migraine includes a preventive component, e.g. the reduction of the frequency of migraine attacks, as well as a rescue component, e.g. the amelioration of the intensity of an acute migraine attack.

Thus, the inventive combination or composition provided herein may be used, for example, for (i) decreasing migraine attack frequency, (ii) decreasing migraine attack duration (iii) decreasing migraine attack severity, and/or (iv) reducing any of the migraine symptoms described herein, e.g.: any of the neurological symptoms associated with migraine, such as phono-, photo-, and/or osmophobia, visual, sensory or motor disturbances, and/or allodynia; and/or any other features known to accompany, precede or follow a migraine attack, such as fatigue, nausea, cognitive difficulties, tiredness, ravenous hunger or thirst, muscle ache, reduced libido, depression, mania, and/or mood swings; and/or preventing, retarding or reversing the transition of episodic migraine to chronic migraine.

Furthermore, the inventive combination or composition provided herein may be useful for reversing, retarding or preventing structural or functional nerve cell damage, such as white matter lesions or disturbances in functional connectivity, associated with migraine.

Furthermore, administration of the inventive combination or composition provided herein may have the effect of decreasing migraine attack frequency, decreasing migraine attack severity, decreasing the severity of migraine symptoms, preventing disease progression and/or preventing disease chronification.

The combination or composition provided herein may be useful for preventing, treating and/or ameliorating chronic headache and/or acute headache. For example, the subject to be treated may have more or less than 5, 10, 15, 20, 25 or 30 headache days per month.

The inventive combination or composition provided herein is particularly useful for treating acute headache and/or an acute migraine attack and/or symptoms of an acute migraine attack.

The symptoms of an acute migraine attack are the same as described herein in the context of migraine in general. However, the treatment of an acute migraine attack refers, in particular, to a rescue treatment. Such a rescue treatment is especially required when the migraine attack cannot be prevented anymore and is about to take its course. Thus, the inventive combination or composition is particularly useful as a rescue treatment for ameliorating or eliminating acute headache, an acute migraine attack and/or symptoms thereof, in particular, for a relevant amount of time. Thus, the treatment of acute headache, an acute migraine attack and/or symptoms thereof, may also comprise the prevention of recurrence of the acute headache, an acute migraine attack and/or symptoms thereof, e.g. for at least about 0.5, 1 or 2 days.

Furthermore, a subject suffering from acute headache may have not suffered from headache for at least about 2, 3, 5, 7, 10 or 14 days of at least about a month.

Furthermore, a subject suffering from acute headache may be not suffering from chronic headache, e.g. the subject suffering from acute headache may not have more than 5, 10, 15, 20, 25 or 30, preferably 15, headache days per month.

Thus, in the context of the present invention, the acute headache and/or acute migraine attack or the symptoms thereof may be ameliorated or eliminated within about 24, 12, 8, 6, 4, 3, 2, or 1 hours or less, preferably within about 2 hours or less. Furthermore, the intensity of the acute headache and/or acute migraine attack or symptoms thereof may be decreased by at least about 50%, 60%, 70%, 80%, 90%, or 100%, e.g. as measured on a visual analogue scale, preferably within about 24, 12, 8, 6, 4, 3, 2, or 1 hours or less. For example, the acute headache and/or acute migraine attack or symptoms thereof may be eliminated within about 2 hours or less. Furthermore, a recurrence of acute headache and/or an acute migraine attack or symptoms thereof may be prevented for at least about 2, 4, 8, 12, 24, 36 or 48 hours, preferably for at least about 12 hours, preferably for at least about 24 hours.

Furthermore, herein and in the context of the present invention, the treatment of acute headache, an acute migraine attack and/or symptoms thereof may include a reduction of the duration of the headache, the acute migraine attack and/or symptoms thereof. For example, the duration of the acute headache, the acute migraine attack and/or symptoms thereof may be reduced by at least about 30%, 50%, 70%, or 90%. Furthermore, the duration of the acute headache, the acute migraine attack and/or symptoms thereof may be reduced by at least 2, 4, 6, 8, 10, 12, 24 or 48 hours.

Furthermore, herein and in the context of the present invention, the treatment of acute headache, an acute migraine attack and/or symptoms thereof may include prevention of an aggravation of the acute headache, an acute migraine attack and/or symptoms thereof. For example, the aggravation of the acute headache or acute migraine attack and/or symptoms thereof may be prevented for at least about 2, 4, 8, 12, 24, 36 or 48 hours, preferably for at least about 12 hours, more preferably for at least about 24 hours.

Furthermore, herein and in the context of the present invention, the treatment of acute headache, an acute migraine attack and/or symptoms thereof may include prevention of the recurrence of aggravated headache and/or an aggravated acute migraine attack or symptoms thereof upon an initial amelioration, e.g. for at least about 2, 4, 8, 12, 24, 36 or 48 hours, preferably for at least about 12 hours, preferably for at least about 24 hours.

Preferably herein, the amelioration of acute headache, an acute migraine attack and/or symptoms thereof, refers to a reduction of the intensity of the headache, as described herein. Preferably, the intensity of the headache is measured on a visual analogue scale, as described herein. In addition, any or all of the symptoms of migraine described herein, e.g. inter alia, nausea, fatigue, tiredness, light, noise and/or smell sensitivity, cognitive difficulties, and/or word finding difficulties may be ameliorated or eliminated.

Preferably herein, the prevention of an aggravation of acute headache, an acute migraine attack and/or symptoms thereof, refers to the suppression of an increase of the intensity of the headache, as described herein.

Preferably, herein and in the context of the present invention, the intensity of the headache and/or migraine is classified based on a visual analogue scale (VAS) that is suitable for measuring the intensity of pain. In the context of the present invention, the pain refers, in particular, to the pain perceived in the head including, inter alia, the brain, skull and face, i.e. “headache”. Furthermore, the perceived intensity of the pain may be influenced by accompanying symptoms that are caused and/or associated with the headache, e.g. further symptoms of migraine, as described herein.

A visual analog scale (VAS) is a tool widely used to measure pain. A patient is asked to indicate his/her perceived pain intensity, e.g. along a 100 mm horizontal line, and this rating is then measured from the left edge (=VAS score), e.g. wherein 10 mm=1 pain point, 0=no pain, and 10=worst pain imaginable. Furthermore, the scale may be accompanied by emoticons which visualize the intensity of pain, e.g. by happy, neutral, sad or suffering faces, and/or colors or symbols which are associated with “good” or “bad”. The VAS is a well-established valid and reliable measure for both acute and chronic pain (see, e.g., Hawker (2011), Arthritis Care Res (Hoboken); 63, Suppl 11).

The same as described herein in the context of “headache” may also apply mutatis mutandis to “pain” as described herein.

The premonitory phase (or prodromal phase), as used herein, is characterized by premonitory symptoms that are the first indications of a migraine attack. These can occur a few hours or several days before the intensity of a migraine attack reaches its peak.

The premonitory phase of a migraine attack can involve a symptomatology, such as changes in cognitive capability, mood changes, frequent yawning, thirst, coldness, increased urinary frequency, difficulty of sleeping or increased tiredness, and/or sensory sensitivities such as photophobia and phonophobia.

The most common premonitory symptoms include: fatigue, mood changes (e.g., depression or irritability), and/or gastrointestinal symptoms (e.g. a change in bowel habits or nausea). Further common premonitory symptoms include: muscle stiffness, aching and/or pain, e.g. in the neck, back and/or face; food cravings or a loss of appetite; difficulties to concentrate; confusion; feeling cold; sensitivity to light, sound, and/or smells; excessive yawning; and/or vivid dreams.

Premonitory symptoms can warn a subject of an impending headache and therefore offer therapeutic potential. In particular, premonitory symptoms can warn a patient of an ongoing attack before the pain phase has started. Furthermore, as illustrated in the appended examples, the combination of a ketone body and/or ketogenic compound and an analgesic and/or antioxidant is particularly effective when the intensity of the headache is still low, e.g. at the end of the premonitory phase.

Preferably herein, and in the context of the present invention, the combination of a ketone body and/or ketogenic compound and the analgesic and/or antioxidant or the inventive composition is to be administered when the migraine and/or symptoms thereof commence, in the premonitory phase of a migraine attack, e.g. at the end of the premonitory phase when the headache begins, and/or during an aura. Thus, said combination or composition may be preferably administered to a subject, when the subject feels that a migraine attack is taking its course, even when no headache is present yet, or when the intensity of the headache is still low, e.g. when the headache is about 0 to about 4 on a visual analogue scale ranging from 0 to 10. In other words, the combination of composition of the invention is preferably administered at the beginning of acute headache, and/or the beginning of an acute migraine attack and/or symptoms thereof (e.g. in the premonitory phase). Thus, in preferred embodiments of the invention, the inventive combination or composition provided herein is to be administered to a subject when the intensity of the headache is between about 0 and about 4, preferably between about 0 and about 3, preferably between about 0 and about 2, preferably about 1, as measured on a visual analogue scale ranging from 0 to 10. Furthermore, the inventive combination or composition provided herein is preferably administered to said subject, when at least one, preferably at least 2, 3, 4, 5, 6, 7, 8, 9 or 10, preferably at least 4, preferably at least 6, of the symptoms selected from the following group of symptoms are present: fatigue; change in mood; a gastrointestinal symptom such as nausea; muscle stiffness and/or pain; food cravings or a loss of appetite; difficulties to concentrate; confusion; feeling cold; sensitivity to light, sound, and/or smells; excessive yawning; and vivid dreams.

In the context of the present invention, the ketone body and/or ketogenic compound may be administered before or after, or at about the same time said analgesic and/or antioxidant is to be administered to the subject. For example, the ketone body and/or ketogenic compound, and said analgesic and/or antioxidant may be administered to the subject with a time difference of at most about 1 h, 30 min, 20 min, 10 min, or 5 min. Preferably, the ketone body and/or ketogenic compound are to be administered at about the same time.

In the context of the present invention, i.e. in the context of the inventive uses of the combinations provided herein, the acetylsalicylic acid may be used at a dose of about 100 to about 2000 mg, preferably about 250 to about 1000 mg, preferably about 500 mg.

Furthermore, in the context of the present invention, i.e. in the context of the inventive compositions and uses thereof, the composition may comprise about 100 to about 2000 mg, preferably about 250 to about 1000 mg, preferably about 500 mg, of acetylsalicylic acid.

Furthermore, in the context of the present invention, i.e. in the context of the inventive uses of the combinations provided herein, the ibuprofen may be used at a dose of about 100 to about 1600 mg, preferably about 200 to about 800 mg, preferably about 400 mg.

Furthermore, in the context of the present invention, i.e. in the context of the inventive compositions and uses thereof, the composition may comprise about 100 to about 1600 mg, preferably about 200 to about 800 mg, preferably about 400 mg, of ibuprofen.

In the context of the present invention, i.e. in the context of the inventive uses of the combinations provided herein, the triptan, e.g. frovatriptan, may be used at a dose of about 1 to about 10 mg, preferably about 2 to about 5 mg, preferably about 2.5 mg. Furthermore, in the context of the present invention, i.e. in the context of the inventive compositions and uses thereof, the composition may comprise about 1 to about 10 mg, preferably about 2 to about 5 mg, preferably about 2.5 mg, of a triptan, e.g. frovatriptan.

In the context of the present invention, i.e. in the context of the inventive uses of the combinations provided herein, melatonin may be used at a dose of about 0.5 to about 20 mg, preferably about 2 to about 15 mg (e.g. about 10 mg), preferably about 9 mg. Furthermore, in the context of the present invention, i.e. in the context of the inventive compositions and uses thereof, the composition may comprise about 0.5 to about 20 mg, preferably about 2 to about 15 mg (e.g. about 10 mg), preferably about 9 mg, of melatonin.

The dose or content of other analgesics and/or antioxidants described herein can be readily adjusted to have a similar efficacy as the doses or contents of acetylsalicylic acid or ibuprofen, frovatriptan or melatonin, provided herein. In particular, the dose or content of other nonsteroidal anti-inflammatory drugs can be readily adjusted to have a similar efficacy as the doses or contents of acetylsalicylic acid or ibuprofen provided herein.

In the context of the present invention, i.e. in the context of the inventive uses of the combinations provided herein, the βHB or β-hydroxybutyrate salt may be used at a dose of about 1 to about 100 g, preferably about 5 to about 40 g, preferably about 10 to about 20 g, preferably about 10 g.

Furthermore, in the context of the present invention, i.e. in the context of the inventive compositions and uses thereof, the composition may comprise about 1 to about 100 g, preferably about 5 to about 40 g, preferably about 10 to about 20 g, preferably about 10 g, of said βHB or β-hydroxybutyrate salt.

The dose or content of other ketone bodies or ketogenic compounds described herein, can be readily adjusted to have a similar efficacy as the doses or contents of the βHB or β-hydroxybutyrate salt provided herein.

In particular, administration of a ketone body and/or ketogenic compound provided herein in the context of the present invention to a subject increases the ketone body concentration, i.e. the βHB concentration, in the blood of said subject, preferably to between about 0.3 mM and about 5 mM, preferably to between about 0.5 mM and about 5 mM, preferably to between about 1 mM and about 3 mM.

Furthermore, administration of a ketone body and/or ketogenic compound provided herein in the context of the present invention to a subject may increase the ketone body concentration, i.e. the βHB concentration, in the blood of said subject at least about 1.5-fold over a corresponding baseline concentration. In particular, the baseline concentration refers to the concentration measured before ingestion of a ketone body or ketogenic compound.

Thus, determining the ketone body concentration, i.e. βHB concentration, in the blood upon administration of a ketone body or ketogenic compound, further allows to determine the dose of a ketone body and/or ketogenic compound that should be used in the context of the inventive uses provided herein or the content of a ketone body and/or ketogenic compound that should be comprised in the inventive compositions provided herein. Hence, the βHB salts used in the appended Examples can be readily replaced with other ketone bodies or ketogenic compounds described herein without any particular difficulties. However, the preferred ketone body or ketogenic compounds to be used in the context of the present invention comprise or consist of βHB.

Preferably, in the context of the present invention, the ketone body concentration in the blood is increased within about 2 h, 1h, 30 min, 15 min or less, preferably within about 30 min or less upon administration of the ketone body or ketogenic compound, or the inventive composition provided herein. Furthermore, the increased ketone body concentration in the blood may be maintained for at least about 1, 2, 4, 6, 8, 10, 12 or 14 hours, preferably for at least about 1 or 2 hours, preferably for at least about 2 hours.

For example, the ketone body and/or ketogenic compound, e.g. the βHB, may be administered to the subject in doses between about 0.01 and about 1 g/kg body weight. For example, the ketone body and/or ketogenic compound, e.g. the βHB, may be administered to the subject once, twice, three times or four times per day.

For example, the daily dose of the ketone body and/or ketogenic compound, e.g. the βHB, to be administered may be about 0.05 g/kg to about 1 g/kg body weight (=about 3.5-70 g/70 kg), about 0.1 g/kg to about 0.7 g/kg body weight (=about 7-49 g/70 kg), or about 0.2 g/kg to about 0.4 g/kg body weight (=about 14-28 g/70 kg). For example, the daily dose of the ketone body and/or ketogenic compound, e.g. the βHB, to be administered may be about 3.5 g to about 70 g, about 5 g to about 50 g, or about 10 g to about 40 g, for example about 10 g, or about 20 g.

Preferably, the ketone body and/or ketogenic compound and/or the analgesic and/or antioxidant, or the inventive composition provided herein is to be administered to the subject orally or parenterally, preferably orally. Thus, the inventive composition provided herein may be formulated for oral or parenteral administration, preferably, oral administration. For example, the ketone body and/or ketogenic compound and/or the analgesic and/or antioxidant, or the inventive composition provided herein may be formulated as a powder for oral administration. Furthermore, the powder or tablet may be formulated for dissolution in water prior to consumption. Furthermore, the powder or tablet, may be formulated for dissolution in the mouth. In some embodiments, the ketone body and/or ketogenic compound and/or the analgesic and/or antioxidant, or the inventive composition provided herein is formulated as a drink.

However, the ketone body and/or ketogenic compound and/or the analgesic and/or antioxidant, or the inventive composition provided herein may be also formulated for other types of administration that are common in the art, e.g. as a suppository for rectal administration, or as an infusion for infusion into the blood.

Preferably herein, i.e. in the context of the treatment of acute headache, an acute migraine attack and/or symptoms thereof, the ketone body and/or ketogenic compound, the analgesic and/or antioxidant, and/or the inventive composition provided herein is formulated to rapidly increase the concentrations of the ketone body and/or analgesic and/or antioxidant in the blood. Thus, the ketone body and/or ketogenic compound, the analgesic and/or antioxidant and/or the inventive composition may be preferably formulated as a powder or tablet for dissolution in an aqueous solution such as water, a powder or tablet for dissolution in the mouth, or as a drink.

Furthermore, ketone body, and/or ketogenic, the analgesic and/or antioxidant and/or the inventive composition provided herein may be formulated for a combined rapid and slow release, e.g. wherein part of said compounds of compositions is embedded or encapsulated in a microparticle as described herein, and the other part is not encapsulated.

Of note, microparticles may be also formulated, inter alia, as a powder or tablet for dissolution in an aqueous solution such as water, a powder or tablet for dissolution in the mouth, or as a drink.

In some embodiments, i.e. in the context of the inventive uses of the combinations provided herein, the ketone body and/or ketogenic compound is formulated as one composition, preferably a pharmaceutical composition, and said analgesic and/or antioxidant is formulated as a separate composition, preferably a pharmaceutical composition.

However, herein and in the context of the present invention, the ketone body and/or ketogenic compound, and said analgesic and/or antioxidant may be also formulated as one composition, preferably a pharmaceutical composition.

The composition of the invention, e.g. the pharmaceutical composition, may be in form of a powder, a tablet, a capsule, a microparticle, a gas, an aerosol, a solution or a suspension. The composition of the invention, e.g. in the context of the inventive food products, may be also in form of a food-grade gummy such as a gummy bear. Furthermore, the composition of the invention, e.g. the pharmaceutical composition, may comprise at least one pharmaceutically acceptable excipient.

Pharmaceutically acceptable excipients are well known in the art and include, interalia, antiadherents such as magnesium stearate, binders such as saccharides (e.g. sucrose, lactose, starches, cellulose, xylitol, sorbitol or mannitol), gelatin, polyvinylpyrrolidone (PVP), or polyethylene glycol (PEG), coatings such as hydroxypropyl methylcellulose, shellac, zein, fatty acids, waxes, plastics, and plant fibers, colors, disintegrants such as crosslinked polyvinylpyrrolidone, crosslinked sodium carboxymethyl cellulose or sodium starch glycolate, flavors (e.g. menthol, citrus, or berry), glidants such as silica gel, fumed silica, talc, or magnesium carbonate, lubricants such as talc, silica, vegetable stearin, magnesium stearate or stearic acid, preservatives such as antioxidants (e.g. vitamin C), cysteine, methionine, citric acid, sodium citrate, methyl paraben or propyl paraben, sorbents, sweeteners (e.g. stevia, saccharin, acesulfame, suclarose), vehicles such as petrolatum, dimethyl sulfoxide or mineral oil, and gummies.

Furthermore, the ketone body and/or ketogenic compound, or the inventive composition, may further comprise a food grade desiccant, i.e. silica (silicon dioxide), e.g., at 4% (w/w) or less, e.g. 3.2 to 3.4% (w/w), e.g. when said ketone body and/or ketogenic compound or composition contains β-hydroxybutyric acid.

The combined content of the ketone body and/or ketogenic compound and the analgesic and/or antioxidant in the inventive composition provided herein may be at least about 20%, 30%, 40%, 50%, 60%, 70%, 80%, 90% or 100% (w/w) of the composition, preferably at least about 50%, preferably at least about 90%.

The mixing ratio of the ketone body and/or ketogenic compound vs. the analgesic and/or antioxidant may be from about 10000:1 to about 1:10000, 1000:1 to about 1:1000, 100:1 to about 1:100, 10:1 to about 1:10, 2:1 to about 1:2, or about 1:1 (weight vs. weight). Preferably, the mixing ratio of the ketone body and/or ketogenic compound, e.g. βHB, vs. the analgesic and/or antioxidant, e.g. acetylsalicylic acid or ibuprofen, may be from about 1000:1 to about 1:2, preferably from about 100:1 to about 5:1, more preferably from about 50:1 to about 10:1, e.g. about 30:1 or about 20:1 (weight vs. weight).

Furthermore, the inventive composition provided herein may be vegan, and/or it may be free of plastic.

Furthermore, in the context of the present invention, the ketone body and/or ketogenic compound, or the inventive composition provided herein may be contained in a microparticle. In particular, said microparticle further comprises a pharmaceutically acceptable matrix. For example, the ketone body and/or ketogenic compound and/or the analgesic and/or antioxidant may be dispersed and/or embedded in the pharmaceutically acceptable matrix. The pharmaceutically acceptable matrix may comprise (i) a denatured and/or polymerized protein such as a pea protein, (ii) a polysaccharide such as an alginate, and/or (iii) a co-polymer of methacrylic acid and methylmethacrylate such as Eudragit. Said denatured and/or polymerized protein may comprise, for example, pea protein, soy bean protein, whey protein, gelatin, or casein, preferably yellow pea protein. Furthermore, the microparticle may be in form of a hydrogel. Said polysaccharide and/or hydrogel may comprise, for example, alginate, agarose, cellulose, starch and/or pectin, preferably an alginate. In particular, the matrix and/or hydrogel may enable a sustained release, controlled release and/or slow release of the ketone body and/or ketogenic compound, and optionally also of the analgesic and/or antioxidant, as described herein. For example, the microparticle may remain intact during gastric transit and a ketone body and/or ketogenic compound, an optionally also the analgesic and/or antioxidant, comprised therein may be released in the small intestine, specifically in the ileum. Preferably, the microparticle has a size of between about 50 μm and about 500 μm, preferably between about 50 μm and about 200 μm. The microparticles may have a variety of structures and shapes. For example, the microparticle may be a microbead and/or have a spherical shape.

Suitable microparticles or microbeads containing a ketone body or ketogenic compound and methods for producing such microparticles or microbeads are described, e.g., in WO2020229364, which is incorporated herein for reference in its entirety. Further reference with respect to the production of microparticles or microbeads is made to WO2016096929A1.

In the context of the present invention, the microparticles or microbeads described in WO2020229364 may further contain an analgesic and/or antioxidant as described herein. Furthermore, these microparticles or microbeads may contain instead of or in addition to a ketone body described in WO2020229364 a ketone body or ketogenic compound described herein in the context of the present invention.

An advantage of using a microparticle is that an increased ketone body blood concentration can be maintained considerably longer compared to other formulations. Thus, a microparticle may be particularly useful for the prevention of headache, a migraine attack and/or symptoms thereof, the prevention of the recurrence of headache, a migraine attack and/or symptoms thereof, and/or the prevention of the recurrence of an aggravated headache, migraine attack and/or symptoms thereof upon an initial amelioration.

Advantageously, enclosing a ketone body or ketogenic compound in a microparticle may further improve the taste of the composition and/or decrease gastrointestinal distress.

A further advantage of enclosing a ketone body or ketogenic compound within a microparticle is to prevent undesired alterations of the physical or chemical properties of the ketone body or ketogenic compound by the environment and/or prevent said ketone body from affecting the environment in an undesired way. In particular, enclosing a ketone body or ketogenic compound in a microparticle may reduce the hygroscopy of the ketone body or ketogenic compound (e.g. β-hydroxybutyric acid), increase the stability of the ketone body or ketogenic compound in an aqueous solution, prevent the degradation of the ketone body or ketogenic compound and/or prevent uptake of the ketone body in an undesired location such as the stomach.

Thus, administration of a microparticle containing a ketone body or ketogenic compound according to the invention to a subject may increase the ketone body concentration in the blood plasma of said subject as described herein, e.g. to between about 0.3 mM and about 5 mM and/or at least about 1.5-fold over a corresponding baseline concentration, and maintain said increased ketone body blood concentration for about 4, 6, 8, 10, 12 or 14 hours, preferably for about 8 or about 12 hours. Furthermore, in context of the invention, the ketone body and/or ketogenic compound and the analgesic and/or antioxidant described herein may be further combined with caffeine. Thus, the inventive composition provided herein may further comprise caffeine.

In some embodiments, the analgesic comprises acetylsalicylic acid or ibuprofen, preferably acetylsalicylic acid, in combination with acetaminophen. In particular, in these embodiments the combination or composition according to the invention may further comprise caffeine.

Furthermore, the ketone body and/or ketogenic compound and the analgesic and/or antioxidant described herein may be combined with further vitamins, co-enzymes, minerals and/or other micronutrients, or nutraceuticals that have been shown to be migraine protective or mitochondrial function enhancing. In particular, the ketone body and/or ketogenic compound and the analgesic and/or antioxidant described herein may be combined with at least 1, 2, 3, 4, 5, 6, 7, 8, 9, 10, 12, 15, 20, further vitamins, co-enzymes, minerals, micronutrients, or nutraceuticals selected from the group consisting of: Vitamin A (e.g. as beta-carotene), Vitamin C (e.g. as calcium ascorbate, ascorbic acid, ascorbyl palmitate, magnesium ascorbate, niacinamide ascorbate, acerola extract), Vitamin D3 (e.g. as cholecalciferol) or D2, Vitamin E (e.g. as mixed tocopherols containing D-alpha tocopheryl succinate, D-alpha tocopherol), Thiamine (vitamin B1) (e.g. as thiamine HCl), Riboflavin (vitamin B2) (as e.g. riboflavin, riboflavin 5′-phosphate), Niacin (as e.g. 61% niacinamide, 38% niacin, 1% niacinamide ascorbate), Vitamin B6 (e.g. as pyridoxal 5′-phosphate, pyridoxine HCl), Folate (e.g. as L-5-methyltetrahydrofolate calcium salt), Vitamin B12 (e.g. as methylcobalamin or adenosylcobalamin), Biotin, Pantothenic acid (e.g. as D-calcium pantothenate with 5 mg pantethine), Calcium (e.g. as dicalcium phosphate, Ca ascorbate, D-calcium pantothenate, Ca D-glucarate), Iodine (e.g. as potassium iodide), Magnesium (e.g. as magnesium oxide, citrate, arginate, glycinate, taurinate, or ascorbate), Zinc (e.g. as zinc citrate, or zinc mono-L-methionine sulfate), Selenium (e.g. as sodium selenite, or Se-methyl L-selenocysteine), Copper (e.g. as copper bisglycinate chelate), Manganese e.g. (as manganese citrate, or gluconate), Chromium, Molybdenum (e.g. as molybdenum amino acid chelate), Potassium (e.g. as potassium citrate), CoQ10 (ubichinon or ubiquinol), L-Lysine, L-Leucine, Broccoli concentrate, N-acetyl-L-cysteine (NAC), Decaffeinated green tea extract (leaf), Acerola Extract, Inositol, Bitter orange citrus bioflavonoids, Fruit/berry blend (e.g. European elder, blackberry, blueberry, sweet cherry, cranberry, plum, persimmon (Diospyros kaki) powders), Taurine, Wild blueberry anthocyanin extract, Ashwagandha extract (root, leaf), Silymarin (from milk thistle extract (seed)), Trimethylglycine, sour cherry (tart cherry) proanthocyanidin powder (skin), pomegranate extract (fruit), Natural mixed tocopherols (providing gamma, delta, alpha, beta tocopherols), bilberry extract (fruit), grape proanthocyanidin extract (whole grape), grape seed proanthocyanidin extract, Quercetin, Bromelain, Lutein, Olive extract (fruit), Sesame seed extract, Luteolin, Apigenin, Boron (e.g. as boron amino acid chelate), Lycopene (e.g. natural tomato extract), and black currant extract.

Thus, the inventive composition provided herein, e.g. the pharmaceutical composition, the food product, food supplement or nutritional aid, may further comprise at least 1, 2, 3, 4, 5, 6, 7, 8, 9, 10, 12, 15, 20, of said vitamins, co-enzymes, minerals, micronutrients, or nutraceuticals.

It is further contemplated that the ketone body and/or ketogenic compound and the analgesic and/or antioxidant described herein may be further combined with a further compound or composition that is suitable for the treatment of pain, headache, migraine and/or symptoms of migraine. Thus, the inventive composition, e.g. the pharmaceutical composition, provided herein may comprise a further compound or composition that is suitable for the treatment of pain, headache, migraine and/or symptoms of migraine.

Furthermore, the invention relates to a kit comprising (I) a ketone body and/or ketogenic compound and an analgesic and/or antioxidant, as described herein, and/or (II) the inventive composition provided herein. The kit may further comprise a brochure or leaflet with information or instructions how to use the ketone body and/or ketogenic compound and the analgesic and/or antioxidant or the composition according to the present invention, or how to carry out the inventive methods provided herein.

Furthermore, the ketone body, ketogenic compound, analgesic, and/or antioxidant may be contained in separate containers, sachets or vials in the kit, or in the same container, sachet or vial. For example, a container, sachet or vial may contain a single dose of the ketone body, ketogenic compound, analgesic, and/or antioxidant or the inventive composition provided herein. Furthermore, the kit may comprise a measuring device such as, inter alia, a spoon or a cup which is suitable for providing a single dose of the ketone body, ketogenic compound and/or analgesic, or the inventive composition provided herein.

Furthermore, the present invention relates to the following items:
1. A combination of (i) a ketone body and/or a ketogenic compound, and (ii) an analgesic, for use in treating and/or preventing headache, migraine and/or symptoms of migraine in a subject.
2. The combination for use according to item 1, wherein said headache, migraine and/or symptoms of migraine are refractory to treatment with acetylsalicylic acid or ibuprofen as single active ingredient.
3. The combination for use according to items 1 or 2, wherein acute headache and/or an acute migraine attack and/or symptoms thereof are treated.
4. The combination for use according to item 3, wherein the acute headache and/or acute migraine attack or the symptoms thereof are ameliorated or eliminated within about 24, 12, 8, 6, 4, 3, 2, or 1 hours or less, preferably within about 2 hours or less.
5. The combination for use according to items 3 or 4, wherein the intensity of the acute headache and/or acute migraine attack or symptoms thereof are decreased by at least about 50%, 60%, 70%, 80%, 90%, or 100%, preferably within about 24, 12, 8, 6, 4, 3, 2, or 1 hours or less.
6. The combination for use according to any one of items 3 to 5, wherein the acute headache and/or acute migraine attack or symptoms thereof are eliminated within about 2 hours or less.
7. The combination for use according to any one of items 4 to 6, wherein a recurrence of acute headache and/or an acute migraine attack or symptoms thereof is prevented for at least about 2, 4, 8, 12, 24, 36 or 48 hours, preferably for at least about 12 hours, preferably for at least about 24 hours.
8. The combination for use according to any one of items 3 to 7, wherein an aggravation of the acute headache and/or acute migraine attack and/or symptoms thereof is prevented.
9. The combination for use according to item 8, wherein the aggravation of the acute headache or acute migraine attack and/or symptoms thereof is prevented for at least about 2, 4, 8, 12, 24, 36 or 48 hours, preferably for at least about 12 hours, preferably for at least about 24 hours.
10. The combination for use according to any one of items 1 to 9, wherein said ketone body and/or ketogenic compound is to be administered before or after, or at about the same time said analgesic is to be administered to said subject.
11. The combination for use according to item 10, wherein said ketone body and/or ketogenic compound, and said analgesic are to be administered to the subject with a time difference of at most about 1 h, 30 min, 20 min, 10 min, or 5 min, preferably at about the same time.
12. A composition comprising (i) a ketone body and/or a ketogenic compound, and (ii) an analgesic.
13. The composition of item 12, wherein said composition is a food product or food supplement.
14. The composition of item 12, wherein said composition is a pharmaceutical composition.
15. The composition of item 12 for use in treating and/or preventing headache, migraine and/or symptoms of migraine in a subject according to any one of items 1 to 11.
16. The combination for use according to any one of items 1 to 11, the composition of any one of items 12 to 14, or the composition for use according to item 15, wherein said ketone body comprises
   (i) βHB: β-hydroxybutyric acid, β-hydroxybutyrate, and/or a pharmaceutically acceptable β-hydroxybutyrate salt, and/or
   (ii) AcAc: acetoacetic acid, acetoacetate, and/or a pharmaceutically acceptable acetoacetate salt, and wherein said ketogenic compound comprises a precursor, preferably a metabolic precursor, of said βHB and/or AcAc.
17. The combination for use according to any one of items 1 to 11 or 16, the composition of any one of items 12 to 14 or 16, or the composition for use according to items 15 or 16, wherein said ketogenic compound is metabolized into βHB and/or AcAc in the body of a subject.
18. The combination for use according to any one of items 1 to 11 or 16 or 17, the composition of any one of items 12 to 14 or 16 or 17, or the composition for use according to any one of items 15 to 17, wherein said ketone body comprises a pharmaceutically acceptable β-hydroxybutyrate salt.
19. The combination or composition for use according to any one of items 16 to 18, or the composition of any one of items 16 to 18, wherein said pharmaceutically acceptable salt is selected from the group consisting of: a potassium salt, a sodium salt, a calcium salt, a magnesium salt, an arginine salt, a zinc salt, a barium salt, an iron salt, a copper salt, a manganese salt, molybdenum salt, a lithium salt, a phosphor salt, a sulfur salt, an iodine salt, a selenium salt, a chromium salt, a cobalt salt, a lysine salt, a leucine salt, a histidine salt, an ornithine salt, a creatine salt, an agmatine salt, a citrulline salt, a methyl glucamine salt and a carnitine salt, or a combination of at least two of said salts.
20. The combination or composition for use according to any one of items 16 to 19, or the composition of any one of items 16 to 19, wherein said pharmaceutically acceptable salt comprises at least 1, 2, 3 or 4, preferably all, salts selected from the group consisting of: a calcium salt, a sodium salt, a potassium salt, and a magnesium salt.
21. The combination or composition for use according to any one of items 16 to 20, or the composition of any one of items 16 to 20, wherein said pharmaceutically acceptable salt is a blend of sodium, calcium, potassium, magnesium mineral salts at a mixing ratio of about 1:2:1:2, about 1:1:1:2 or about 1:1:1:1.
22. The combination or composition for use according to any one of items 16 to 21, or the composition of any one of items 16 to 21, wherein said ketone body comprises β-hydroxybutyric acid.
23. The combination or composition for use according to any one of items 16 to 22, or the composition of any one of items 16 to 22, wherein said ketone body comprises a pharmaceutically acceptable β-hydroxybutyrate salt and β-hydroxybutyric acid.
24. The combination for use according to any one of items 1 to 11 or 16 to 23, the composition of any one of items 12 to 14 or 16 to 23, or the composition for use according to any one of items 15 to 22, wherein said ketone body comprises D-β-hydroxybutyrate and/or D-β-hydroxybutyric acid, and/or said ketogenic compound is metabolized into D-β-hydroxybutyrate and/or D-β-hydroxybutyric acid.
25. The combination for use according to any one of items 1 to 11 or 16 to 24, the composition of any one of items 12 to 14 or 16 to 24, or the composition for use according to any one of items 15 to 24, wherein said ketone body does not comprise L-β-hydroxybutyrate and/or L-β-hydroxybutyric acid, and/or said ketogenic compound is not metabolized into L-β-hydroxybutyrate and/or L-β-hydroxybutyric acid.
26. The combination for use according to any one of items 1 to 11 or 16 to 25, the composition of any one of items 12 to 14 or 16 to 25, or the composition for use according to any one of items 15 to 25, wherein said ketogenic compound comprises a metabolic precursor of βHB and/or AcAc selected from the group consisting of: 1,3-butanediol, triacetin, a fatty acid, e.g. a medium chain fatty acid, or a compound comprising a fatty acid such as a medium chain triglyceride, or a ketogenic amino acid such as leucine or lysine; or a pharmaceutically acceptable salt of said metabolic precursor.
27. The combination or the composition for use according to any one of items 1 to 11 or 16 to 26, the composition of any one of items 12 to 14 or 16 to 26, or the composition for use according to any one of items 15 to 26, wherein said ketogenic compound comprises a compound comprising an acetoacetyl- and/or 3-hydroxybutyrate moiety.
28. The combination for use according to any one of items 1 to 11 or 16 to 27, the composition of any one of items 12 to 14 or 16 to 27, or the composition for use according to any one of items 15 to 27, wherein said ketogenic compound comprises a ketone ester, or a pharmaceutically acceptable salt thereof.
29. The combination or composition for use according to item 28 or the composition of item 28, wherein said ketone ester comprises an ester of βHB and/or AcAc.
30. The combination or composition for use according to items 28 or 29 or the composition of items 28 or 29, wherein said ketone ester comprises an ester formed by the union of βHB with βHB, βHB with AcAc, or AcAc with AcAc.
31. The combination or composition for use according to any one of items 28 to 30 or the composition of any one of items 28 to 30, wherein said ketone ester comprises an ester formed by the union of βHB or AcAc with a monohydric, dihydric or trihydric alcohol.
32. The combination or composition for use according to any one of items 28 to 31, or the composition of any one of items 28 to 31, wherein said ketone ester comprises an ester formed by the union of βHB or AcAc with 1,3-butanediol.
33. The combination or composition for use according to any one of items 28 to 32, or the composition of any one of items 28 to 32, wherein said ketone ester comprises an ester formed by the union of βHB or AcAc with glycerol, for example, glyceryl tris(3-hydroxybutyrate).
34. The combination or composition for use according to any one of items 28 to 33, or the composition of any one items 28 to 33, wherein said ketone ester comprises an ester formed by the union of βHB with 1,3-butanediol or glycerol, for example a (R)-β-Hydroxybutyrate-R-1,3-butanediol monoester.
35. The combination or composition for use according to items 33 or 34, or the composition of any one items 28 to 34, wherein said glycerol is further esterified with at least one fatty acid, e.g. a medium chain fatty acid.
36. The combination or composition for use according to any one of items 28 to 35, or the composition of any one of items 28 to 35, wherein said ketone ester comprises an ester formed by the union of 1,3-butanediol with at least one fatty acid, e.g. a medium chain fatty acid, for example, 1,3-butanediol esterified with two caproic acids.
37. The combination or composition for use according to any one of items 28 to 36, or the composition of any one of items 28 to 36, wherein said ketone ester is an ester of D-βHB and/or said ketone ester is metabolized in the body of a subject into D-βHB and/or acetoacetate, preferably at least D-βHB.
38. The combination for use according to any one of items 1 to 11 or 16 to 37, the composition of any one of items 12 to 14 or 16 to 37, or the composition for use according to any one of items 15 to 37, wherein said ketogenic compound comprises an amide of βHB and/or AcAc, or a pharmaceutically acceptable salt thereof.
39. The combination for use according to any one of items 1 to 11 or 16 to 38, the composition of any one of items 12 to 14 or 16 to 38, or the composition for use according to any one of items 15 to 38, wherein said ketogenic compound comprises a compound selected from the group consisting of: D-beta-hydroxybutyrate-D-1,3-butanediol; (3R)-hydroxybutyl-(3R)-hydroxybutyrate; acetoacetyl-1 ,3-butanediol; acetoacetyl-R-3-hydroxybutyrate; acetoacetylglycerol; 3-hydroxybutyl 3-hydroxybutanoate; (3-hydroxy-1-methyl-propyl) 3-hydroxybutanoate; 3-(3-hydroxybutanoyloxy)butyl 3-hydroxybutanoate; 3-(3-hydroxybutanoyloxy)butanoic acid; 3-hydroxybutyl 3-oxobutanoate; 3-hydroxy-1-methyl-propyl) 3-oxobutanoate; 3-(3-oxobutanoyloxy)butyl 3-oxobutanoate; 3-(3-oxobutanoyloxy)butanoic acid; 2,3-dihydroxypropyl 3-oxobutanoate; [2-hydroxy-1-(hydroxymethyl)ethyl]3-oxobutanoate; [2-hydroxy-3-(3-oxobutanoyloxy)propyl]3-oxobutanoate; [3-hydroxy-2-(3-oxobutanoyloxy)propyl]3-oxobutanoate; 2,3-bis(3-oxobutanoyloxy)propyl 3-oxobutanoate; and any one of the formulae

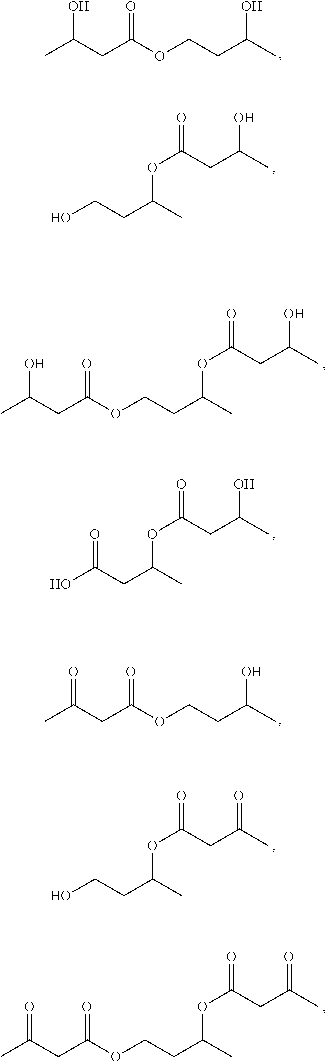

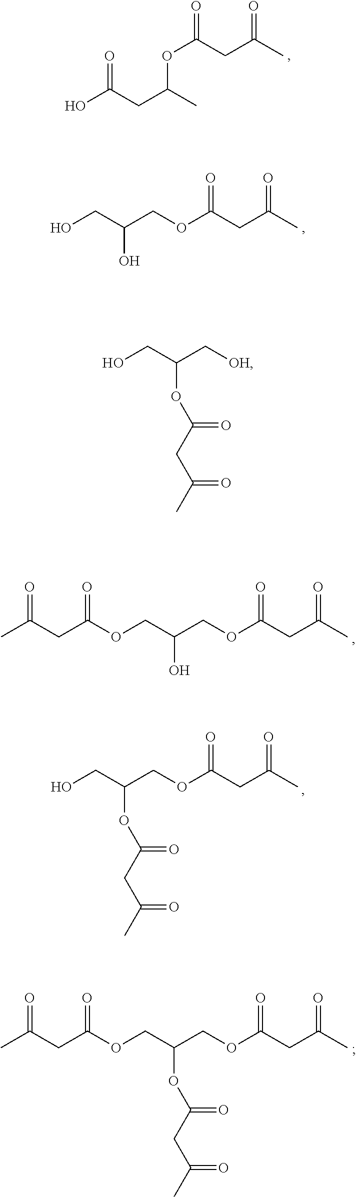

and a pharmaceutically acceptable salt thereof.
40. The combination for use according to any one of items 1 to 11 or 16 to 39, the composition of any one of items 12 to 14 or 16 to 39, or the composition for use according to any one of items 15 to 39, wherein said analgesic comprises (i) a nonsteroidal anti-inflammatory drug, (ii) an inhibitor of cyclooxygenase, preferably at least of COX2, (iii) acetaminophen, (iv) a triptan, (v) melatonin, (vi) an opioid and/or (vii) a cannabioid.
41. The combination for use according to any one of items 1 to 11 or 16 to 40, the composition of any one of items 12 to 14 or 16 to 40, or the composition for use according to any one of items 15 to 40, wherein said analgesic comprises at least one nonsteroidal anti-inflammatory drug selected from the group consisting of the following (i) to (vii):
   (i) a salicylate such as acetylsalicylic acid, diflunisal (e.g. Dolobid), salicylic acid or a salt thereof, or salsalate (e.g. Disalcid);
   (ii) a propionic acid derivative such as ibuprofen, dexibuprofen, naproxen, fenoprofen, ketoprofen, dexketoprofen, flurbiprofen, oxaprozin, loxoprofen, pelubiprofen, or zaltoprofen;
   (iii) an acetic acid derivative such as diclofenac, indomethacin, tolmetin, sulindac, etodolac, ketorolac, aceclofenac, bromfenac, or nabumetone;
   (iv) an enolic acid (e.g. oxicam) derivative such as piroxicam, meloxicam, tenoxicam, droxicam, lornoxicam, or phenylbutazone (e.g. Bute);
   (v) an anthranilic acid derivative (fenamate) such as mefenamic acid, meclofenamic acid, flufenamic acid, or tolfenamic acid;
   (vi) a selective COX-2 inhibitor (coxib) such as celecoxib, parecoxib, or etoricoxib; and
   (vii) clonixin, licofelone or H-harpagide.
42. The combination for use according to any one of items 1 to 11 or 16 to 41, the composition of any one of items 12 to 14 or 16 to 41, or the composition for use according to any one of items 15 to 41, wherein said analgesic comprises a salicylate and/or a propionic acid derivative such as ibuprofen, dexibuprofen, dexketoprofen or naproxen.
43. The combination for use according to any one of items 1 to 11 or 16 to 42, the composition of any one of items 12 to 14 or 16 to 42, or the composition for use according to any one of items 15 to 42, wherein said analgesic comprises acetylsalicylic acid and/or ibuprofen.
44. The combination for use according to any one of items 1 to 11 or 16 to 43, the composition of any one of items 12 to 14 or 16 to 43, or the composition for use according to any one of items 15 to 43, wherein said ketone body and/or ketogenic compound comprises a pharmaceutically acceptable β-hydroxybutyrate salt, and said analgesic comprises acetylsalicylic acid or ibuprofen.
45. The combination or composition for use according to items 43 or 44, or the composition of items 43 or 44, wherein the acetylsalicylic acid is used at a dose of about 100 to about 2000 mg, preferably about 250 to about 1000 mg, preferably about 500 mg, or wherein said composition comprises about 100 to about 2000 mg, preferably about 250 to about 1000 mg, preferably about 500 mg, of the acetylsalicylic acid.
46. The combination or composition for use according to any one of items 43 to 45, or the composition of any one of items 43 to 35, wherein the ibuprofen is used at a dose of about 100 to about 1600 mg, preferably about 200 to about 800 mg, preferably about 400 mg, or wherein said composition comprises about 100 to about 1600 mg, preferably about 200 to about 800 mg, preferably about 400 mg, of the ibuprofen.
47. The combination or composition for use according to any one of items 44 to 46, or the composition of any one of items 44 to 46, wherein said βHB or β-hydroxybutyrate salt is used at a dose of about 1 to about 100 g, preferably about 5 to about 40 g, preferably about 10 to about 20 g, or wherein said composition comprises about 1 to about 100 g, preferably about 5 to about 40 g, preferably about 10 to about 20 g of said βHB or β-hydroxybutyrate salt.
48. The combination for use according to any one of items 1 to 11 or 16 to 47, the composition of any one of items 12 to 14 or 16 to 47, or the composition for use according to any one of items 15 to 47, wherein administration of said ketone body and/or ketogenic compound or said composition to a subject increases the ketone body concentration, i.e. the βHB concentration, in the blood of said subject to between about 0.3 mM and about 5 mM, preferably to between about 0.5 mM and about 5 mM, preferably to between about 1 mM and about 3 mM.
49. The combination for use according to any one of items 1 to 11 or 16 to 48, the composition of any one of items 12 to 14 or 16 to 48, or the composition for use according to any one of items 15 to 48, wherein administration of said ketone body and/or ketogenic compound or said composition to a subject increases the ketone body concentration, i.e. the βHB concentration, in the blood of said subject at least about 1.5-fold over a corresponding baseline concentration.
50. The combination or composition for use according to items 48 or 49, or the composition of items 48 or 49, wherein the ketone body concentration in the blood is increased within about 2 h, 1h, 30 min, 15 min or less, preferably within about 30 min or less.
51. The combination or composition for use according to any one of items 48 to 50, or the composition of any one of items 48 to 50, wherein said ketone body blood concentration is maintained for at least about 1, 2, 4, 6, 8, 10, 12 or 14 hours, preferably at least about 1 or 2 hours, preferably at least about 2 hours.
52. The combination for use according to any one of items 1 to 11 or 16 to 51 or the composition for use according to any one of items 15 to 51, wherein said combination, composition, or ketone body and/or ketogenic compound is to be administered to said subject in doses between about 0.01 and about 1 g/kg body weight and/or once, twice, three times or four times per day.
53. The combination for use according to any one of items 1 to 11 or 16 to 52, the composition of any one of items 12 to 14 or 16 to 52, or the composition for use according to any one of items 15 to 52, wherein said combination, composition, or ketone body and/or ketogenic compound is to be administered to said subject orally, or wherein said composition is formulated for oral administration.
54. The combination for use according to any one of items 1 to 11 or 16 to 53, the composition of any one of items 12 to 14 or 16 to 53, or the composition for use according to any one of items 15 to 53, wherein
   (i) said ketone body and/or ketogenic compound is formulated as a pharmaceutical composition, and said analgesic is formulated as a separate pharmaceutical composition; or
   (ii) said ketone body and/or ketogenic compound, and said analgesic are formulated as one pharmaceutical composition, wherein the pharmaceutical composition may be in form of a powder, a tablet, a capsule, a microparticle, a solution or a suspension, and/or further comprise a pharmaceutically acceptable excipient.
55. The combination for use according to any one of items 1 to 11 or 16 to 54, the composition of any one of items 12 to 14 or 16 to 54, or the composition for use according to any one of items 15 to 54, wherein said ketone body and/or ketogenic compound, or said composition is contained in a microparticle, wherein said microparticle further comprises a pharmaceutically acceptable matrix.
56. The combination or composition for use according to item 55, or the composition of item 55, wherein said pharmaceutically acceptable matrix comprises (i) a denatured and/or polymerized protein such as a pea protein, (ii) a polysaccharide such as an alginate, and/or (iii) a co-polymer of methacrylic acid and methylmethacrylate such as Eudragit.
57. The combination or composition for use according to any one of items 54 to 56, or the composition of any one of items 54 to 56, wherein said microparticle has a size of between about 50 μm and about 500 μm, preferably between about 50 μm and about 200 μm.
58. The combination or composition for use according to any one of items 54 to 57, or the composition of any one of items 54 to 57, wherein administration of said microparticle to a subject increases the ketone body concentration in the blood plasma of said subject to between about 0.3 mM and about 5 mM and/or at least 1.5-fold over a corresponding baseline concentration, and maintains said increased ketone body blood concentration for about 4, 6, 8, 10, 12 or 14 hours, preferably for about 8 or about 12 hours.
59. The combination for use according to any one of items 1 to 11 or 16 to 58, the composition of any one of items 12 to 14 or 16 to 58, or the composition for use according to any one of items 15 to 58, wherein said combination or composition further comprises caffeine.
60. The combination for use according to any one of items 1 to 11 or 16 to 59, or the composition for use according to any one of items 15 to 59, wherein said migraine or symptoms of migraine comprise headache.
61. The combination for use according to any one of items 1 to 11 or 16 to 60, or the composition for use according to any one of items 15 to 60, wherein said headache is unilateral.
62. The combination for use according to any one of items 1 to 11 or 16 to 61, or the composition for use according to any one of items 15 to 61, wherein said headache is throbbing or pulsating.
63. The combination for use according to any one of items 1 to 11 or 16 to 62, or the composition for use according to any one of items 15 to 62, wherein said headache is of moderate to high intensity, e.g. about 5 to about 10 on a visual analogue scale ranging from 0 to 10.
64. The combination for use according to any one of items 1 to 11 or 16 to 63 or the composition for use according to any one of items 15 to 63, wherein said migraine and/or symptoms thereof comprise headache, and at least one, preferably at least 2, 3, 4, 5, 6, 7, 8, 9, or 10, further symptom(s) selected from the group consisting of: an aura; nausea, sickness or vomiting; light, noise and/or smell sensitivity; balance disturbance or loss of balance; word finding difficulties; sensory and/or motor disturbances; allodynia; cognitive difficulties such as lightheadedness, brain fog, and/or difficulties to focus or concentrate; confusion; frequent yawing; changes in appetite and food craving such as binge eating; tiredness, fatigue or low energy; mood changes, e.g. from depression to euphoria; increased thirst and urination; neck stiffness; facial pain; head tension; sore eyes; changes in temperature sensation such as feeling too hot or too cold; diarrhea or constipation; sweating; irritability, dizziness or feeling faint.
65. The combination for use according to any one of items 1 to 11 or 16 to 64 or the composition for use according to any one of items 15 to 64, wherein said migraine and/or symptoms thereof comprise headache and at least one further symptom selected from the group consisting of: an aura; nausea, sickness and/or vomiting; and light, noise and/or smell sensitivity.
66. The combination for use according to any one of items 1 to 11 or 16 to 65 or the composition for use according to any one of items 15 to 65, wherein headache and at least one, preferably at least 2, 3, 4, 5 or all symptoms selected from nausea, fatigue, tiredness, light, noise and/or smell sensitivity, cognitive difficulties, and word finding difficulties are ameliorated or eliminated.
67. The combination for use according to any one of items 1 to 11 or 16 to 66 or the composition for use according to any one of items 15 to 66, wherein said subject has previously suffered from at least one migraine attack and/or is suffering from recurrent migraine attacks.
68. The combination for use according to any one of items 1 to 11 or 16 to 67 or the composition for use according to any one of items 15 to 67, wherein said subject is suffering from depression, mania, mood swings, and/or a reduced libido.
69. The combination for use according to any one of items 1 to 11 or 16 to 68 or the composition for use according to any one of items 15 to 68, wherein said combination or composition is to be administered when the subject has an aura, when the migraine and/or symptoms thereof commence, and/or in the premonitory phase of the migraine attack, in particular when the intensity of headache is still low or the headache is still absent, e.g. about 0 to about 4 on a visual analogue scale ranging from 0 to 10.

The invention is also characterized by the following figures, figure legends and the following non-limiting examples.

### BRIEF DESCRIPTION OF THE DRAWINGS

FIG. 1. A ketone body and an analgesic ameliorate headache during acute migraine attacks in a migraine patient in a synergistic way. Shown is the average headache intensity (pain) during 11 migraine attacks in a migraine patient upon the medication as indicated, as measured on a visual analogue scale ranging from 0 to 10. The 0h time-point indicates the time of medication intake. The ketone body was a mix of β-hydroxybutyrate mineral salts (MigraKet), and the analgesic was either acetylsalicylic acid or ibuprofen (i.e. a simple analgesic). MigraKet alone: n=3; acetylsalicylic acid or ibuprofen alone: n=4; MigraKet in combination with acetylsalicylic acid or ibuprofen: n=4.

FIG. 2. Scheme of proof-of-concept clinical trial. The unmarked arrows indicate measurement of blood ketone and glucose levels from home using a KetoMojo portable finger prick device at baseline and 30 min and 60 min after medication consumption. The arrows marked by a star indicate the time-points of questionnaires (migraine frequency, medication use, intensity, migraine disability, adverse events, migraine related disability, tolerability, etc.). The arrow marked with a triangle indicates the final inclusion or exclusion, study medication and KetoMojo and stripes disposal. The arrow marked with a hexagon indicates the return of empty and full study medication sachets. V3* and V4* are virtual visits.

### EXAMPLES

Methods and materials are described herein for use in the present disclosure; other, suitable methods and materials known in the art can also be used. The materials, methods, and examples are illustrative only and not intended to be limiting.

### Example 1. Improved Treatment of Acute Migraine Attacks by a Combination of (i) βHB and (ii) a Simple Analgesic, i.e. Acetylsalicylic Acid or Ibuprofen, a Triptan, or Melatonin

A female migraine patient who had a history of recurrent migraine attacks and deemed to be a “metabolic migraineur” (with more than 50% of her migraines being triggered by a metabolic event, such as fasting, skipping a meal, exercise or similar) was selected for this initial trial. During the course of the trial, the patient had 17 recorded acute migraine attacks which were treated by either one of the following nine treatment regimes:
1. 13 g flavoured mix of β-hydroxbutyrate (βHB) mineral salts termed “MigraKet” (a ketone body)
2. 500 mg acetylsalicylic acid (a simple analgesic)
3. 400 mg ibuprofen (a simple analgesic)
4. 13 g MigraKet+500 mg acetylsalicylic acid (combination of a ketone body and a simple analgesic)
5. 13 g MigraKet+400 mg ibuprofen (combination of a ketone body and a simple analgesic)
6. 2.5 mg frovatriptan (a triptan)
7. 13 g MigraKet+2.5 mg frovatriptan (combination of a ketone body and a triptan)
8. 9 mg melatonin
9. 13 g MigraKet+9 mg melatonin (combination of a ketone body and an antioxidant such as melatonin)
   The “MigraKet” consisted of:

10g of a sodium, potassium, magnesium and calcium D-βHB mineral mix in a 1:1:2:2 ratio, and 3g of flavour (malic acid and natural lemon and lime flavour sweetend by stevia), dissolved in 0.51 of still water.

The intensity of the headache (pain) was recorded at the time of intake of the substance(s) (0h), and at five subsequent time-points during the course of one day (2h, 4 h, 8h, 12 h, 24h after substance intake). The intensity of the headache (pain) was categorized on a visual analogue scale from 0 (“no pain”) to 10 (“worst pain imaginable”). The observations are shown in Table 1.

TABLE 1
Intensity of the headache during an acute migraine attack at the
time of intake of the substance(s) and during the following 24 h

Intensity of the headache (pain)

| Treatment regime | 0 h | 2 h | 4 h | 8 h | 12 h | 24 h |
|---|---|---|---|---|---|---|
| MigraKet | 1 | 0 | 0 | 3 | 6 | 6 |
| Acetylsalicylic acid | 4 | 3 | 4 | 6 | 8 | 8 |
| Ibuprofen | 4 | 2 | 3 | 7 | 9 | 8 |
| Acetylsalicylic acid | 2 | 0 | 0 | 2 | 4 | 6 |
| Ibuprofen | 2 | 0 | 0 | 0 | 3 | 5 |
| MigraKet | 2 | 2 | 2 | 3 | 5 | 6 |
| MigraKet + | 2 | 0 | 0 | 0 | 0 | 0 |
| Acetylsalicylic acid | | | | | | |
| MigraKet + | 2 | 0 | 0 | 0 | 0 | 0 |
| Ibuprofen | | | | | | |
| MigraKet | 4 | 5 | 8 | 8 | 9 | 7 |
| MigraKet + | 1 | 0 | 0 | 0 | 0 | 0 |
| Acetylsalicylic acid | | | | | | |
| MigraKet + | 6 | 4 | 4 | 6 | 7 | 7 |
| Acetylsalicylic acid | | | | | | |
| Frovatriptan | 2 | 0 | 0 | 0 | 3 | 5 |
| MigraKet + | 2 | 0 | 0 | 0 | 0 | 0 |
| Frovatriptan | | | | | | |
| Melatonin | 2 | 2 | 2 | 4 | 8 | 8 |
| Melatonin | 1 | 2 | 4 | 6 | 6 | 6 |
| MigraKet + | 1 | 1 | 0 | 0 | 0 | 0 |
| Melatonin | | | | | | |
| MigraKet + | 2 | 2 | 2 | 2 | 4 | 5 |
| Melatonin | | | | | | |

For further evaluation, the treatment regimes were grouped into the following three groups:
1. MigraKet (ketone body only)
2. Acetylsalicylic acid or ibuprofen (simple analgesic only)
3. MigraKet+acetylsalicylic acid or ibuprofen (combination of ketone body and simple analgesic)

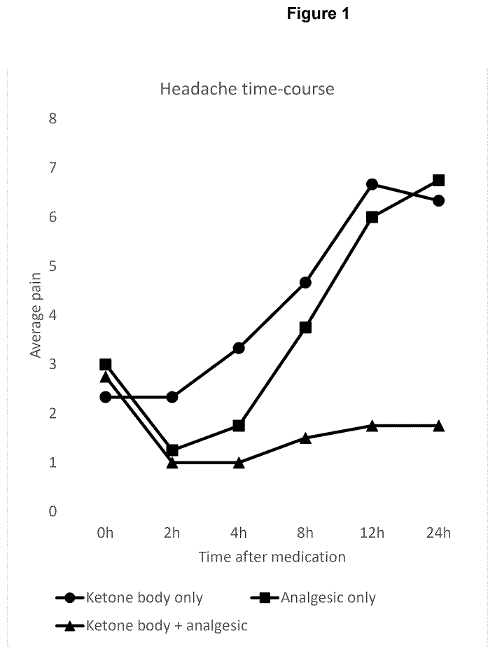

The average intensities for the three groups at the various time-points are shown in FIG. 1.

The intensity of the headache at the beginning of the treatment regime, i.e. the time of substance intake (0h), was similar between the three groups (-2-3). No significant differences were found between acetylsalicylic acid alone and ibuprofen alone. Surprisingly, however, the combination of βHB with acetylsalicylic acid or ibuprofen decreased the intensity of the headache for at least 24h, whereas βHB, acetylsalicylic acid or ibuprofen alone ameliorated the headache, in average, very little or only transiently, i.e. for only about 4-8 hours.

Hence, these data suggest that the combination of a ketone body or a ketogenic compound (e.g. βHB) and an analgesic (e.g. acetylsalicylic acid or ibuprofen) have a synergistic effect for the treatment of headache during acute migraine attacks. Moreover, it was surprisingly found that the combination of βHB with acetylsalicylic acid or ibuprofen completely eliminated the headache already within at most 2 hours and prevented the recurrence of the headache until the end of the experiment (24h) in 3 out of 4 instances, which is very uncommon when treating a migraine attack with a pain modulating substance, i.e. an analgesic, only. In contrast, βHB alone only eliminated the headache in 1 out of 3 instances, and acetylsalicylic acid or ibuprofen alone only in 2 out of 4 instances. Moreover, βHB, acetylsalicylic acid or ibuprofen alone never prevented the recurrence of the headache during the course of the experiment, but at best, only for about 4-12 hours.

It was further observed that even a triptan (e.g. frovatriptan) alone eliminated the headache for only less than 12h, whereas the combination of a triptan with βHB eliminated the headache for at least 24h. This suggests that the combination of a ketone body and/or ketogenic compound with a triptan is advantageous over the individual components alone.

Furthermore, it was observed that melatonin alone did not ameliorate the headache, which got severely aggravated over the course of 24 hours, whereas in 1 out of 2 instances, the combination of melatonin with βHB eliminated the headache for at least 20h. In the second instance, the combination of melatonin with βHB at least prevented a strong aggravation of the headache (only a mild aggravation starting after 8h was observed). This suggests that the combination of a ketone body and/or ketogenic compound with melatonin is advantageous over the individual components, i.e. melatonin, alone.

Furthermore, in the only instance, when the combination of βHB with acetylsalicylic acid or ibuprofen did not ameliorate or eliminate the headache, the intensity of the pain was already relatively high (“6”) at the beginning of the treatment (at the intake of the substance(s)). This further suggests that the underlying cause of the migraine attack can be tackled or ameliorated best when the attack is not yet full blown. However, even in that instance, the combination, at least, prevented a further aggravation of the headache.

These data further indicate that the combination of a ketone body or ketogenic compound and an analgesic is preferably administered at the beginning of an acute migraine attack, i.e. when the headache is still low (e.g about 0 to about 4 on a visual analogue scale from 0 to 10) and/or in the premonitory phase, to achieve an optimal effect.

### Example 2. Proof-of-Concept Clinical Trial

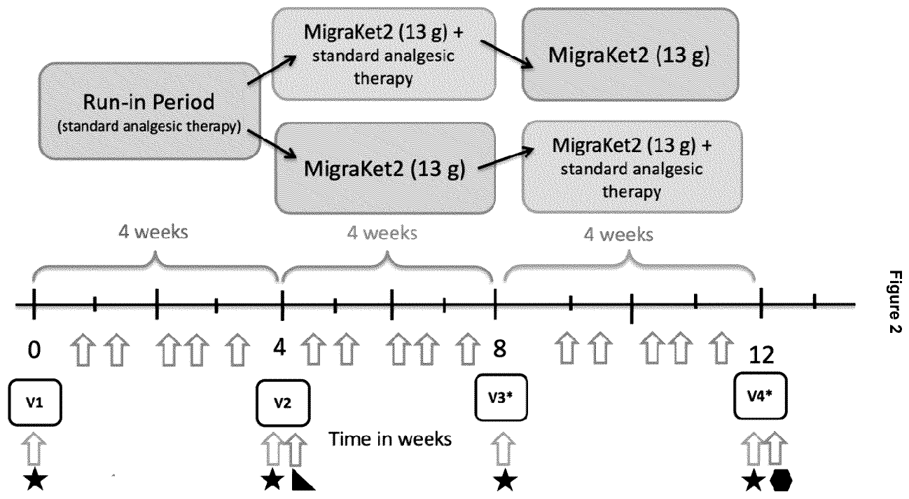

The following clinical trial is envisaged, interalia, to further confirm the effects observed with a migraine patient described in Example 1. In particular, the clinical trial is carried out to confirm that a ketone body or ketogenic compound, e.g. MigraKet, is useful for the treatment of acute migraine attacks. Moreover, the clinical trial is also carried out to confirm an additive or synergistic effect of the addition of a ketone body or ketogenic compound, e.g. βHB, to a standard acute migraine treatment, i.e. treatment with an analgesic such as acetylsalicylic acid or ibuprofen.

| | |
|---|---|
| Title of clinical trial | Long title: Tolerability and efficacy of MigraKet for acute |
| | treatment of migraine: A proof-of concept study |
| | Short title: MigraKet in acute migraine treatment (MigraKet |
| | PoC study) |
| Clinical trial type and | An open-label proof of concept trial |
| phase | |
| Objective(s) | To test if exogenous beta-hydroxybutyrate in mineral salt |
| | form (MigraKet) alone or in combination with the patient's |
| | standard analgesic acute migraine therapy, i.e. an |
| | analgesic such as acetylsalicylic acid or ibuprofen, taken |
| | before or at migraine pain (headache) onset (e.g. at |
| | earliest premonitory symptom), compared to baseline, |
| | reduces migraine pain, duration of attack and number of |
| | analgesics or triptans used in metabolic episodic |
| | migraineurs (5-14 migraine days/month) by at least 25% |
| | and which dose is doing that most effectively (assessed by |
| | a headache diary for clinical endpoints). |
| Intervention(s) | Experimental interventions: |
| | 1 13 g MigraKet (comprising 10 g Mg—Ca—Na—K D-beta- |
| | hydroxybutyrate powder (taken orally) - If there is |
| | no migraine relief 2 hours after MigraKet ingestion, |
| | standard rescue medication may be taken. |
| | 2 13 g MigraKet (consisting of roughly 10 g Mg—Ca—Na—K |
| | D-beta-hydroxybutyrate powder (taken orally) |
| | plus standard acute therapy, e.g. an analgesic such |
| | as acetylsalicylic acid or ibuprofen |
| | Control interventions: |
| | None |
| | Duration of baseline per patient: 4 weeks |
| | Duration of intervention per patient: 2 × 4 weeks |
| | Total trial duration per patient: 3 months |
| | See FIG. 2 for the trial scheme. |
| Key inclusion and | Key inclusion criteria: Diagnosis of episodic migraine (5- |
| exclusion criteria | 14 migraine days/month, 18-65 years old, stable |
| | preventive treatment for >3 months; at least 2 metabolic |
| | migraine trigger factors: Skipping a meal/fasting, |
| | Exercise, Hypoxia/altitude, Dehydration, Alcohol, |
| | Psychological or physical stress) |
| | Key exclusion criteria: Any significant neurological, |
| | psychiatric or other medical condition, Botox within 6 |
| | months of study onset, anti-inflammatory drug (NSAIDs) or |
| | triptan use greater than 10 days per month, current |
| | participation in other migraine trial |
| Endpoint(s) | Exploratory endpoints: (to better inform following phase 2b |
| | trial): |
| | Pain/migraine intensity (VAS 0-10) 2 h-12 h |
| | after taking study drug (in 2 h intervals, unless |
| | interrupted by sleep) |
| | Change from baseline in days with migraine |
| | during 4 weeks of intervention |
| | Reduction in acute migraine medication |
| | (analgesics or triptans) |
| | Reduction in duration of the migraine attacks/ |
| | Time to next migraine attack |
| | Reduction in associated migraine symptoms |
| | Change in migraine Disability Assessment |
| | Change in headache days of any severity |
| | Possible further exploratory endpoint(s): |
| | Peak ketone body and blood glucose level change |
| | from baseline (Measured 0, 30 min and 60 min after |
| | ingestion) and a KetoMojo device |
| | tolerability |
| Sample size | Number of patients to be assessed for eligibility: 30 |
| | Number of patients to be allocated to the trial: 25 |
| | Number of patients to be analysed: 20 |
| Statistical analysis | Efficacy: After testing for normality either repeated |
| | measures ANOVA or Wilcoxon signed rank test of |
| | endpoints are used. |
| Trial duration | First patient in to last patient out (months): |
| | 6 |
| | (allowing 2 participant batches à 10 patients and rolling |
| | recruitment) |
| | Recruitment period (months; rolling recruitment): |
| | 2 |
| | Duration of the entire trial (months): 6 |
| Participating centres | Single or multi-centre |
| Key words | Migraine, acute treatment, open-label, clinical trial, |
| | exogenous ketone bodies, metabolism |

Furthermore, a placebo-controlled phase 2 trial, with several arms is envisaged.

1-48. (canceled)

49. A method of treating, preventing and/or dietary management of pain, migraine and/or symptoms of migraine and/or supporting brain health in a subject, comprising administering to the subject an effective amount of a combination of (i) a ketone body and/or a ketogenic compound, and (ii) an analgesic and/or an antioxidant.

50. The method of claim 49, wherein said pain is associated with and/or caused by migraine, fibromyalgia, cluster headache and/or rheumatoid arthritis, and/or comprises headache.

51. The method of claim 49, wherein said pain, migraine and/or symptoms of migraine are refractory to treatment with at least one analgesic selected from the group consisting of acetylsalicylic acid, ibuprofen and a triptan as single active ingredient.

52. The method of claim 49, wherein acute headache and/or an acute migraine attack and/or symptoms thereof are treated.

53. The method of claim 49, wherein
(I) said ketone body comprises
(i) β-hydroxybutyric acid, β-hydroxybutyrate, and/or a pharmaceutically acceptable β-hydroxybutyrate salt, collectively referred to as “βHB”, and/or
(ii) acetoacetic acid, acetoacetate, and/or a pharmaceutically acceptable acetoacetate salt, collectively referred to as “AcAc”; and wherein
(II) said ketogenic compound comprises a metabolic precursor of said βHB and/or AcAc selected from the group consisting of:
ketone esters, 1,3-butanediol, triacetin, medium chain triglycerides, and ketogenic amino acids.

54. The method of claim 49, wherein the ketogenic compound is a ketone ester selected from the group consisting of: esters formed by the union of βHB with βHB, βHB with AcAc, or AcAc with AcAc; esters formed by the union of βHB with a fatty acid; esters formed by the union of βHB or AcAc with a monohydric, dihydric or trihydric alcohol, wherein said di- or trihydric alcohol may be further esterified with a fatty acid; and esters formed by the union of 1,3-butanediol with at least one fatty acid.

55. The method of claim 49, wherein said analgesic comprises (i) a nonsteroidal anti-inflammatory drug, (ii) an inhibitor of cyclooxygenase, (iii) acetaminophen, (iv) a triptan, (v) melatonin or a melatonin receptor agonist, (vi) an opioid and/or (vii) a cannabinoid.

59. The method of claim 49, wherein said ketone body and/or ketogenic compound comprises β-hydroxybutyric acid, β-hydroxybutyrate, and/or a pharmaceutically acceptable β-hydroxybutyrate salt.

57. The method of claim 49, wherein said analgesic and/or antioxidant is an analgesic comprising at least one nonsteroidal anti-inflammatory drug.

58. The method of claim 49, wherein said analgesic and/or antioxidant comprises acetylsalicylic acid, ibuprofen, melatonin and/or a triptan.

59. The method of claim 49, wherein said ketone body and/or ketogenic compound comprises β-hydroxybutyric acid, β-hydroxybutyrate, and/or a pharmaceutically acceptable β-hydroxybutyrate salt, and wherein said analgesic and/or antioxidant comprises acetylsalicylic acid and/or ibuprofen.

60. The method of claim 49, wherein said ketone body and/or ketogenic compound comprises β-hydroxybutyric acid, β-hydroxybutyrate, and/or a pharmaceutically acceptable β-hydroxybutyrate salt, and wherein said analgesic and/or antioxidant comprises a triptan.

61. The method of claim 49, wherein said ketone body and/or ketogenic compound comprises D-β-hydroxybutyrate and/or D-β-hydroxybutyric acid.

62. The method of claim 49, wherein said ketone body and/or ketogenic compound comprises β-hydroxybutyric acid, β-hydroxybutyrate, and/or a pharmaceutically acceptable β-hydroxybutyrate salt, and wherein said analgesic and/or antioxidant comprises melatonin and/or a melatonin receptor agonist.

63. The method of claim 49, wherein said migraine or symptoms of migraine comprise unilateral and/or throbbing or pulsating headache, and at least one further symptom selected from the group consisting of: (i) an aura; (ii) nausea, sickness and/or vomiting; and (iii) light, noise and/or smell sensitivity.

64. The method of claim 49, wherein administration of said ketone body and/or ketogenic compound to the subject increases the βHB concentration in the blood of said subject to between about 0.3 mM and about 5 mM.

65. A composition comprising (i) a ketone body and/or a ketogenic compound, and (ii) melatonin and/or a melatonin receptor agonist.

66. The composition of claim 65, wherein said ketone body and/or ketogenic compound comprises β-hydroxybutyric acid, β-hydroxybutyrate, and/or a pharmaceutically acceptable β-hydroxybutyrate salt.

67. A food product, food supplement or nutritional aid, comprising the composition of claim 65.

68. A medical food or food for special medical purposes (FSMP), comprising the composition of claim 65.

69. A pharmaceutical composition comprising the composition of claim 65 and at least one pharmaceutically acceptable excipient.

70. A composition comprising (i) a ketone body and/or a ketogenic compound, and (ii) a triptan.

71. The composition of claim 70, wherein said ketone body and/or ketogenic compound comprises β-hydroxybutyric acid, β-hydroxybutyrate, and/or a pharmaceutically acceptable β-hydroxybutyrate salt.
